(19) United States
(12) Patent Application Publication
(10) Pub. No.: US 2020/0181347 A1
(43) Pub. Date: Jun. 11, 2020

(54) MULTIFUNCTIONAL HYBRID AEROGELS

(71) Applicants: RUTGERS, THE STATE UNIVERSITY OF NEW JERSEY, New Brunswick, NJ (US); STATE UNIVERSITY OF MARINGÁ, Maringá-PR (BR)

(72) Inventors: Tewodros ASEFA, Kendall Park, NJ (US); Heveline Dal Magro FOLLMANN, Maringá (BR); Rafael DA SILVA, Maringá (BR)

(21) Appl. No.: 16/704,993

(22) Filed: Dec. 5, 2019

Related U.S. Application Data
(60) Provisional application No. 62/775,685, filed on Dec. 5, 2018.

(51) Int. Cl. C08J 9/00; A61K 47/02; A61K 47/32

(52) U.S. Cl. CPC C08J 9/008; A61K 47/02; C08J 2433/02; C08J 9/0061; C08J 2329/04; A61K 47/32

(57) ABSTRACT

The present invention relates in part to the unexpected discovery of novel hybrid organic-inorganic aerogel materials with one-dimensionally aligned pores.

### CROSS-REFERENCE TO RELATED APPLICATION

The present application claims priority under 35 U.S.C. § 119(e) to U.S. Provisional Patent Application No. 62/775,685, filed Dec. 5, 2018, which application is incorporated herein by reference in its entirety.

### BACKGROUND OF THE INVENTION

Inorganic nanoporous materials have found applications in many areas, especially as heterogeneous catalysts, adsorbents, membranes, solid-state platforms for solar cells, and so forth. Nanoporous materials have also been used as scaffolds for tissue engineering and as hard templates to build complex engineered nanostructures suitable for biological applications, such as for example controlled drug-delivery. However, the diffusion of bioactive guest molecules through designed nanoporous materials needs to be finely tuned in order to control the release profiles of the guest species in or from them. Further, the nanoporous host materials need to be manipulated to allow for high loading of guest molecules therein.

Aerogels are highly porous, low-density solid-state materials with large internal surface areas. They are synthesized from gel precursors by replacing their liquid components with void spaces. Several techniques can be used to remove the liquid components from gel precursors to make aerogels, and the techniques include supercritical drying, vacuum drying, microwave drying, freeze drying, and ambient pressure drying. In an ideal aerogel synthesis, the liquid component has to be slowly removed from the gel precursor without causing the resulting solid network to collapse due to capillary action.

Aerogels can be made with different compositions, including carbon, organic-inorganic hybrid materials, transition metal oxides, and silica. Owing to their interesting structural features and surface properties, aerogel materials can be used as adsorbents, catalysts, and/or drug delivery systems. Aerogels made of silica, in particular, have special physical properties, including extremely low density (ca. 0.1 g/cm³), low thermal conductivity (12-15 mW/mK), high porosity (>95%), and large specific surface areas (800-1,000 m²/g). In addition, since silica can be chemically modified with simple procedures by taking advantage of its reactive surface hydroxyl groups, the properties of silica-based aerogels can easily be further tailored. Although many of these structural features are also found in mesoporous silica materials, silica aerogels generally comprise freestanding materials with macroscopic sizes (often with dimensions in centimeter-to-meter scales), while mesoporous silicas often constitute micro- or nano-particles. Another important difference between mesoporous silica materials and silica aerogels is that the pore sizes and shapes in silica aerogels can be random. This sometimes allows for their pores to be more accessible, whereas those of mesoporous silicas are often uniform and well organized, and thus relatively less accessible.

Nanoporous silicas and other related materials with high drug loading capacity have an ability to improve the stability and bioavailability of drugs; hence, they are intensively pursued for drug delivery applications. However, silica materials, especially in micro- and nano-particulate forms, can be toxic, which limits their utility.

There is thus a need in the art for novel multifunctional hybrid aerogels. The present invention addresses this unmet need in the art.

### BRIEF SUMMARY OF THE INVENTION

The invention provides a composition comprising poly(vinyl alcohol) (PVA), poly(acrylic acid) (PAA), and at least one mesoporous silica nanoparticle. The invention further provides a method of treating a disease or disorder in a subject using compositions of the invention.

In certain embodiments, the PVA and the PAA are at least partially polymerized.

In certain embodiments, the at least partially polymerized PVA and PAA form a hyperbranched polymer network.

In certain embodiments, the composition is essentially free of solvents or liquid components.

In certain embodiments, the composition is an aerogel.

In certain embodiments, the at least one mesoporous silica nanoparticle is dispersed within the polymer.

In certain embodiments, the at least one mesoporous silica nanoparticle comprises a SBA-15 mesoporous silica nanoparticle.

In certain embodiments, the at least one mesoporous silica nanoparticle is derivatized with at least one functional group.

In certain embodiments, the at least one functional group comprises a hydrophobic group or a quaternary ammonium group.

In certain embodiments, the hydrophobic group comprises a hydrocarbyl, tris(hydrocarbyl)silane, halogen, or thiohydrocarbyl, wherein each group is optionally substituted with at least one selected from hydrocarbyl, tri(hydrocarbyl)silane, halogen, and thiohydrocarbyl.

In certain embodiments, the hydrophobic group is located within a pore of the nanoparticle.

In certain embodiments, the quaternary ammonium group is located on the outer surface of the nanoparticle.

In certain embodiments, the composition further comprises a therapeutic agent that is associated with the at least one functional group.

In certain embodiments, the method comprises administering to the subject an aerogel composition comprising a co-polymer of PVA and PAA, wherein at least one mesoporous silica nanoparticle is dispersed within the co-polymer and is derivatized with the at least one functional group.

In certain embodiments, the at least one functional group is a quaternary ammonium salt. In certain embodiments, the at least one functional group is on the outer surface of the nanoparticle. In certain embodiments, the disease or disorder comprises bacterial infection. In certain embodiments, a therapeutic agent is associated with the at least one functional group. In certain embodiments, the therapeutic agent treats the disease or disorder. In certain embodiments, the at least one functional group is hydrophobic. In certain embodiments, the at least one functional group is within a pore of the nanoparticle. In certain embodiments, the therapeutic agent is hydrophobic.

In certain embodiments, the aerogel composition is administered through a route selected from the group consisting of oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.

### BRIEF DESCRIPTION OF THE DRAWINGS

The following detailed description of selected embodiments of the invention will be better understood when read in conjunction with the appended drawings. For the purpose of illustrating the invention, specific embodiments are illustrated in the drawings. It should be understood, however, that the invention is not limited to the precise arrangements and instrumentalities of the embodiments illustrated in the drawings.

FIGS. 1A-1C are non-limiting schematic illustration of a synthetic procedure leading to silica-polymer hybrid (SPH) aerogels for sustained and prolonged delivery of hydrophobic drugs. First, a solution containing polymers and mesoporous silica nanoparticles is prepared. Next, the solution is frozen. Finally, the frozen material is subjected to sublimation to remove water and form an aerogel. By varying the relative amounts of constituents in the precursor and the synthetic conditions, different SPH materials for drug delivery applications are obtained.

FIGS. 2A-2B illustrate (FIG. 2 A) digital images of SHP-1 before and after treatment at 160° C. for 45 min and (FIG. 2B) schematic representation of the process leading to aerogels comprising crosslinked PAA and PVA with organic-functionalized mesoporous silica nanoparticles entrapped in the polymer matrices.

FIGS. 3A-3H illustrate SEM images of SPH-1 (FIGS. 3A-3B), SPH-2 (FIGS. 3C-3D), SPH-3 (FIGS. 3E-3F), and SPH-4 (FIGS. 3G-3H) aerogels. The image in the inset in FIG. 3B shows some mesoporous silica nanoparticles bulged out of the polymer. See FIGS. 11 and 12 for additional SEM images.

FIGS. 4A-4B illustrate bulk density versus S/L ratio (FIG. 4A) and temporal stability with constant water uptake by the beads as a function of time (FIG. 4B) of different aerogels.

FIG. 5 illustrates adsorption profiles of SPH-4 and SPH-4P for dexamethasone (DEX) fitted with the Freundlich model (solid curves).

FIGS. 6A-6C illustrate release profiles of DEX from DEX-loaded SPH aerogels over several days (FIG. 6A), over the first 10 hours (FIG. 6B), and the portion with zero-order kinetics (FIG. 6C).

FIG. 7 illustrates cell viability of Vero (red bar) and L929 fibroblast (line bar) cells determined by MTT assay after 72 h exposure to functionalized-mesoporous silica nanoparticles and different aerogel materials with a concentration of 200 μg/mL. The corresponding results for the control experiment are also included. The standard deviations were obtained from based on three measurements (n=3).

FIG. 8 illustrates FTIR spectrum of SPH-1 aerogel: before (a) and after (b) immersing the material into aqueous NaOH solution (0.1 M) and then taking it out of the solution and letting it to dry under ambient condition.

FIG. 9 illustrates FTIR spectra of SPH-1 (a), SPH-2 (b), SPH-3 (c), and SPH-4 (d) aerogels after being immersed in aqueous NaOH solution (0.1 M) and then taking them out of the solution and letting them dry under ambient condition.

FIG. 10 illustrates different SPH aerogels dipped into aqueous NaOH solutions (0.1 M). The sample labels go in accordance with those described in Table 1; for example, sample 1C (the second one in the image) corresponds to SPH-1 aerogel, and so on.

FIG. 11 illustrates (left panels) SEM images of SPH-1 hybrid aerogel, and (right panels) SEM images of methyl-functionalized SBA-15 mesoporous silica nanoparticles.

FIG. 12 illustrates SEM images of SPH-1 (A), SPH-2 (B), SPH-3 (C), and SPH-4 (D) aerogels.

FIGS. 13A-13B illustrate adsorption isotherm of DEX in SPH-4 (FIG. 13A) and SPH-4P (FIG. 13B). The adsorption isotherms are obtained using the Langmuir (solid lines) and the Freundlich (dashed lines) models.

FIG. 14 illustrates N₂ adsorption/desorption isotherms of SBA-15 mesoporous silica nanoparticles whose internal pores are functionalized with HMDS using 2-propanol as a solvent (A) and using toluene as a solvent (B). (C) N₂ adsorption/desorption isotherms of SPH-1 to SPH-6 aerogels.

FIGS. 15A-15B illustrate pore size distributions of SBA-15 mesoporous silica nanoparticles whose internal pores are functionalized with HMDS (FIG. 15A) using toluene as a solvent and (FIG. 15B) using 2-propanol as a solvent.

FIG. 16 illustrates optical microscopy of Vero cells after 72 h of incubation with different aerogel materials and a control. Magnification: 100-fold increase in blue filter.

FIG. 17 illustrates TGA traces of SBA-15 mesoporous silica nanoparticles whose internal pores are functionalized with HMDS using 2-propanol as a solvent (black line) and using toluene as a solvent (red line).

### DETAILED DESCRIPTION OF THE INVENTION

The present invention relates in part to the unexpected discovery of novel hybrid organic-inorganic aerogel materials with one-dimensionally aligned pores. In certain embodiments, the materials of the invention can hold a large payload of hydrophobic drugs. In other embodiments, the materials of the invention can release the hydrophobic drug in a sustained and prolonged manner.

Biomaterials often suffer from general toxicity problems. One potential way to avoid or ameliorate biomaterials toxicity is to introduce biocompatible groups in or around the biomaterials, e.g., by functionalizing the materials' surfaces with biocompatible polymers. Polymers are among the systems that are widely used to render biocompatibility to mesoporous silica nanoparticles. Polymers are also commonly used as structural constituents with other systems (for instance, with cellulose nanofibrils) to make hybrid aerogels, which can serve as superadsorbents. In particular, stable branched polymers, which can be made by increasing the extent of their cross-linking and by reducing the degree of freedom of the bonds in their chains, are quite suitable as structural components to make aerogel materials from.

Poly(vinyl alcohol) (PVA) and poly(acrylic acid) (PAA), which are water-soluble polymers, can react with each other via an esterification reaction between PVA's hydroxyl groups and PAA's carboxylic acid groups and form strongly crosslinked polymers. In addition, PVA and PAA are among the most suitable biomaterials and good candidate polymers to serve as components of hybrid aerogels for biological and medical applications due to their multifunctionality as well as nontoxicity/biocompatibility. For instance, PVA displays minimal cell adhesion and protein absorption, and is thus suitable for certain applications in body fluids. Although several hybrid aerogels for drug delivery applications have been reported before, almost all of them suffer from low adsorption capacity for hydrophobic drugs and/or fast drug release profiles for the encapsulated drugs. These properties make such materials unsuitable for some applications (e.g., severe burn treatment).

As demonstrated herein, the present invention provides novel mesoporous silica/hyperbranched hybrid aerogels with good adsorption capacity and prolonged release properties for hydrophobic drugs. In certain embodiments, these aerogels are synthesized from mesoporous silica, PVA, and PAA using the freeze-drying technique, followed by solid-state reaction. The synthetic technique is simple, cost-effective, and environmentally friendly, and allows for the fabrication of inorganic/organic hybrid aerogels with controlled porous structures. In the aerogels the biocompatible, cross-linked PAA- and PVA-based polymers exist as a continuous phase, wherein SBA-15 mesoporous silica nanoparticles, whose internal pore walls and external surfaces are pre-functionalized with hydrophobic moieties and quaternary groups, respectively, are dispersed. Because of the hydrophilicity of their polymer chains and the large number of hydrophobic mesoporous silica nanoparticles in them, the hybrid aerogels can carry a large payload (log P=1.83) of dexamethasone (DEX), a hydrophobic drug with anti-inflammatory and immunosuppressive activity, and then slowly release the drug over long time, with distinctive release profiles depending on the aerogels' exact composition. Notably, also, the hybrid aerogels show no toxicity and the nanoparticles dispersed into the materials present bactericidal character. Due to their hydrophobic pores, the hybrid aerogels show excellent drug loading capacity for DEX, with encapsulation efficiency higher than 75%. Furthermore, the release pattern of the payloads of DEX encapsulated in the aerogels is highly tailorable (i.e., it can be made faster or slower, as needed) simply by varying the PVA-to-PAA weight ratio in the precursors, and thus the 3-dimensional (3-D) structures of the cross-linked polymers in them. The materials also show sustained drug release, for over more than 50 days. Also, the nanoparticles functionalized with quaternized groups and dispersed within the aerogels display bactericidal activity against E. coli, S. aureus, B. subtilis, and P. aeruginosa. Owing to these properties and their ability of prolonged drug release, these aerogels are of particular interest especially for applications such as wound dressing of severe burn injuries.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, a disease or disorder is “alleviated” if the severity of a symptom of the disease or disorder, the frequency with which such a symptom is experienced by a patient, or both, is reduced.

As used herein, the terms “effective amount,” “pharmaceutically effective amount,” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation.

As used herein, the term “effective amount” of a delivery vehicle refers to an amount sufficient of the delivery vehicle to effectively entrap, bind or deliver a compound.

As used herein, the term “hydrogel” or “aquagel” refers to a network of oligomers or polymer chains that are water-insoluble, sometimes found as a colloidal gel in which water is the dispersion medium. Hydrogels can generally absorb much fluid and, at equilibrium, typically are composed of 60-90% fluid and only 10-30% polymer. In certain embodiments, the water content of hydrogel is about 70-80%. Hydrogels may be prepared by crosslinking hydrophilic biopolymers or synthetic polymers. Examples of the hydrogels formed from physical or chemical crosslinking of hydrophilic biopolymers include but are not limited to, hyaluronans, chitosans, alginates (including alginate sulfate), collagen, dextran, pectin, carrageenan, polylysine, gelatins or agarose. Examples of hydrogels based on chemical or physical crosslinking synthetic polymers include but are not limited to HEMA, methacrylate-oligolactide-PEO-oligolactide-methacrylate, poly(ethylene glycol) (PEG), poly(propylene glycol) (PPG), PEG-PPG-PEG copolymers (Pluronics), poly(phosphazene), poly(methacrylates), poly(N-vinylpyrrolidone), PL(G)A-PEG-PL(G)A copolymers, or poly(ethylene imine).

As used herein, the term “hydrophobic group” refers to a group that has affinity for non-polar solvents, such as but not limited to n-octane, over polar solvents, such as but not limited to water. Non-limiting examples of hydrophobic groups include a hydrocarbyl [such as alkyl (for example, C₁-C₁₀₀ alkyl, C₁-₅₀ alkyl, C₁-C₂₀ alkyl, C₁-C₁₀ alkyl, or C₁-C₆ alkyl), cycloalkyl (for example, C₃-C₁₀₀ cycloalkyl, C₃-C₅₀ cycloalkyl, C₃-C₂₀ cycloalkyl, C₃-C₁₀ cycloalkyl, or C₃-C₆ cycloalkyl), carbocyclyl (for example, 3-12 membered carbocyclyl, 3-8 membered carbocyclyl, or 3-6 membered carbocyclyl), alkenyl (for example, C₂-C₁₀₀ alkenyl, C₂-C₅₀ alkenyl, C₂-C₂₀ alkenyl, C₂-C₁₀ alkenyl, or C₂-C₆ alkenyl), alkynyl (for example, C₂-C₁₀₀ alkynyl, C₂-C₅₀ alkynyl, C₂-C₂₀ alkynyl, C₂-C₁₀ alkynyl, or C₂-C₆ alkynyl), and/or aryl (such as but not limited to phenyl and naphthyl)], silane comprising three independently selected hydrocarbyls as defined herein (trihydrocarbylsilane indicates a silane substituted with three independently selected hydrocarbyls), halogen (F, Cl, Br, I), thiohydrocarbyl, and the like; wherein each group is optionally substituted with at least one selected from hydrocarbyl, thiohydrocarbyl, halogen, and trihydrocarbylsilane.

As used herein, the term “hyperbranched” polymer refers to a highly branched three-dimensional (3D) polymeric structure in which all bonds converge to a focal point or core, and which have a multiplicity of reactive chain-ends. Hyperbranched have one or more central atom(s) as branching points to which two or more polymer chains built up by respective repeating monomer units, i.e. arms are attached. These arms contain additional branching points, offering the possibility for further branching and/or cross-linking upon continued polymerization. Hyperbranched polymers can be prepared using various methodologies, such as but not limited to step-growth polycondensation of ABₓ (x≥2) or A₂+B₃ monomers, self-condensing vinyl polymerization, and ring opening polymerization. In certain non-limiting embodiments, hyperbranched polymers can be distinguished from dendrimers. A dendrimer consists of two types of structural units: uniform terminal units on the globular surface and dendritic units inside, and thus well-defined molecular weights with unique symmetric structures. On the other hand, a hyperbranched polymer comprises a mixture of linear and branched units inside, with multifunctional groups on their periphery. As dendrimers, hyperbranched polymers show a highly branched architecture with a three-dimensional globular shape, with termini located on the periphery. However, the structure of hyperbranched polymers is irregular, with linear and branched units randomly distributed within the macromolecular framework (or polymer backbone). Hyperbranched polymers have more irregular structures with polydispersity of molecular weight, unlike dendrimers, with their perfect structures with monodispersity of molecular weight.

As used herein, an “instructional material” includes a publication, a recording, a diagram, or any other medium of expression that can be used to communicate the usefulness of a composition or method of the invention in the kit for treating, preventing or alleviating various diseases or disorders recited herein. Optionally, or alternately, the instructional material can describe one or more methods of treating, preventing or alleviating diseases or disorders in a cell or a tissue of a mammal. The instructional material of the kit of the invention can, for example, be affixed to a container that contains the identified composition or delivery system of the invention or be shipped together with a container that contains the identified composition or delivery system. Alternatively, the instructional material can be shipped separately from the container with the intention that the instructional material and the composition be used cooperatively by the recipient.

As used herein, the term “mesoporous” refers to a material containing pores with diameters between 2 and 50 nm. For comparison, microporous material has pores smaller than 2 nm in diameter and macroporous material has pores larger than 50 nm in diameter. Typical mesoporous materials include some kinds of silica and alumina that have similarly-sized mesopores. Mesoporous oxides of niobium, tantalum, titanium, zirconium, cerium and tin have also been reported, as well as mesoporous carbon, which has direct applications in energy storage devices. A mesoporous material can be disordered or ordered in a mesostructure.

As used herein, the term “nanoparticle” refers to a microscopic particle with at least one dimension between 1 and 100 nanometres (nm) in size.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, trifluoroacetic acid, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, ammonium salts, metallic salts including alkali metal, alkaline earth metal and transition metal salts such as, for example, calcium, magnesium, potassium, sodium, and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine), and procaine. All of these salts may be prepared from the corresponding compound by reacting, for example, the appropriate acid or base with the compound.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any animal, or cells thereof whether in vitro or in situ, amenable to the methods described herein. In a non-limiting embodiment, the patient, subject, or individual is a human.

As used herein, the term “polymer” refers to a molecule composed of repeating structural units typically connected by covalent chemical bonds. The term “polymer” is also meant to include the terms copolymer and oligomers. In one embodiment, a polymer comprises a backbone (i.e., the chemical connectivity that defines the central chain of the polymer, including chemical linkages among the various polymerized monomeric units) and a side chain (i.e., the chemical connectivity that extends away from the backbone).

As used herein, the term “polymerization” or “crosslinking” refers to at least one reaction that consumes at least one functional group in a monomeric molecule (or monomer), oligomeric molecule (or oligomer) or polymeric molecule (or polymer), to create at least one chemical linkage between at least two distinct molecules (e.g., intermolecular bond), at least one chemical linkage within the same molecule (e.g., intramolecular bond), or any combinations thereof. A polymerization or crosslinking reaction may consume between about 0% and about 100% of the at least one functional group available in the system. In one embodiment, polymerization or crosslinking of at least one functional group results in about 100% consumption of the at least one functional group. In other embodiments, polymerization or crosslinking of at least one functional group results in less than about 100% consumption of the at least one functional group.

As used herein, the term “reaction condition” refers to a physical treatment, chemical reagent, or combination thereof, which is required or optionally required to promote a reaction. Non-limiting examples of reaction conditions are electromagnetic radiation, heat, a catalyst, a chemical reagent (such as, but not limited to, an acid, base, electrophile or nucleophile), and a buffer.

As used herein, the term “therapeutic” treatment refers to a treatment administered to a subject who exhibits signs of pathology, for the purpose of diminishing or eliminating those signs.

As used herein, the term “treating a disease or disorder” means reducing the frequency with which a symptom of the disease or disorder is experienced by a patient. Disease and disorder are used interchangeably herein.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “alkyl,” by itself or as part of another substituent means, unless otherwise stated, a straight or branched chain hydrocarbon having the number of carbon atoms designated (i.e. C₁-₆ means one to six carbon atoms) and including straight, branched chain, or cyclic substituent groups. Examples include methyl, ethyl, propyl, isopropyl, butyl, isobutyl, tent-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “haloalkyl” means alkyl as defined above, substituted by one, two or three substituents selected from the group consisting of F, Cl, Br, and I.

As used herein, the term “cycloalkyl” refers to a mono cyclic or polycyclic non-aromatic radical, wherein each of the atoms forming the ring (i.e. skeletal atoms) is a carbon atom. In certain embodiments, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following moieties:

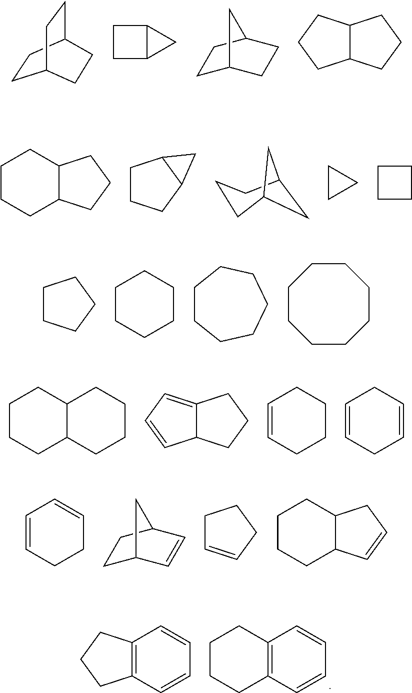

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. Preferred examples are phenyl and naphthyl, most preferred is phenyl.

The term “carbocyclyl” refers to a saturated or unsaturated carbocyclic ring system containing one or more rings (typically one, two or three rings). In certain embodiments, the carbocyclyl is a 3-12 membered carbocyclic ring, a 3-8 membered carbocyclic ring, or a 3-6 membered carbocyclic ring.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. In certain embodiments, a hydrocarbyl comprises only carbon and hydrogen atoms.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two.

As used herein, the term “optionally substituted” means that the referenced group may be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

Throughout this disclosure, various aspects of the invention can be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 and the like, as well as individual numbers within that range, for example, 1, 2, 2.7, 3, 4, 5, 5.3, and 6. This applies regardless of the breadth of the range.

### Description

The present invention relates in part to the unexpected discovery of novel hybrid organic-inorganic aerogel materials with one-dimensionally aligned pores. In certain embodiments, the compositions of the invention contemplate mesoporous silica nanoparticles embedded with certain (co)polymers. In certain embodiments, the materials of the invention can hold a large payload of hydrophobic drugs. In certain embodiments, the materials of the invention can release the hydrophobic drug in a sustained and prolonged manner.

The materials of the invention can be prepared using methods known in the art, and/or using illustrative methods as described elsewhere herein. For example, mesoporous silica nanoparticles can be prepared by polymerizing a silicate (such as but not limited to an orthosilicate, such as but not limited to tetraalkyl orthoslicate, such as but not limited to tetramethyl orthosilicate, tetraethyl orthosilicate, tetrapropyl orthosilicate, tetrabutyl orthosilicate, tetraamyl orthosilicate, tetrahexyl orthosilicate, tetraisopropyl orthosilicate, tetraoctyl orthosilicate, tetrakis (2-ethyl-1-butyl) orthosilicate, and the like) in the presence of an acid, such as hydrogen chloride, hydrogen bromide, sulfuric acid, and the like. The polymerization can take place in an aqueous or non-aqueous system, and the reaction can be performed in the presence of a soluble amphiphilic polymer, such as but not limited to a block copolymer, such as but not limited to a di- or tri-block copolymer, such as but not limited to a pluronic (poloxamer). Without wishing to be limited by any theory, the polymerization of the orthosilicate takes place in such a manner that the pores of the formed mesoporous silica nanoparticles are at least partially occupied by the soluble amphiphilic polymer. The formed material can be isolated from the system through filtration and/or precipitation and/or concentration and purified as needed.

In certain embodiments, amphiphilic polymer-containing mesoporous silica nanoparticle can have its outer surface at least partially derivatized. The outer surface of the nanoparticle comprises reactive hydroxyl groups, which can be reacted with an alkylating, silylating, or acylating agent to introduce functional groups on the outer surface of the nanoparticle. Without wishing to be limited by any theory, the presence of the soluble amphiphilic polymer within the pores of the nanoparticle prevents or minimizes derivatization within the pores of the nanoparticle (i.e., the amphiphilic polymer protects the surface of the pore from derivatization). In certain embodiments, the alkylating, silylating, or acylating agent comprises a quaternary ammonium group (e.g., an aliphatic amine comprising four non-H substituents or an alkylated pyridium group), whereby reaction of the alkylating, silylating, or acylating agent with the hydroxyl groups on the outer surface of the nanoparticle introduces a quaternary ammonium group on the outer surface of the nanoparticle. In other embodiments, the alkylating agent is a chloride, bromide, iodide, triflate, mesylate, and the like. In yet other embodiments, the acylating agent is an acyl chloride, acyl bromide, acyl anhydride, and the like. In yet other embodiments, the silylating agent is a trialkoxysilicate, such as but not limited to TOSPTA.

In certain embodiments, the amphiphilic polymer can be at least partially removed from the pores of the mesoporous silica nanoparticle using methods known in the art, such as but not limited to solvent extraction. An amphiphilic solvent and/or a mixture of a more polar solvent and a more apolar solvent can be used for that extraction.

In certain embodiments, the amphiphilic polymer-free mesoporous silica nanoparticle can have the surface of its pores at least partially derivatized. The surface of the pores comprises reactive hydroxyl groups, which can be reacted with an alkylating, silylating, or acylating agent to introduce functional groups within the pores of the nanoparticle. In certain embodiments, the alkylating, silylating, or acylating agent comprises a hydrophobic group (e.g., an alkyl group or a trialkylsilyl group), whereby reaction of the alkylating, silylating, or acylating agent with the hydroxyl groups in the pore of the nanoparticle introduces a hydrophobic group within the pore of the nanoparticle. In other embodiments, the alkylating agent is a chloride, bromide, iodide, triflate, mesylate, and the like. In yet other embodiments, the acylating agent is an acyl chloride, acyl bromide, acyl anhydride, and the like. In yet other embodiments, the silylating agent is a hexaalkyldisilazane, such as but not limited to hexamethyldisilazane (which allows for the capping of the hydroxyl group with a trimethylsilyl group).

The nanoparticles, which can be optionally derivatized as described elsewhere herein, can be mixed with PVA, PAA, and solvent (such as but not limited to water, water-containing solvents, or water-free solvents). The resulting mixture can be subjected to a process that allows for forming an aerogel, such as but not limited to supercritical drying, vacuum drying, microwave drying, freeze drying, and/or ambient pressure drying. The resulting aerogel can then be subjected to conditions that allow for at least partial polymerization of the PVA and PAA.

The polymers of the invention can be prepared by heating the corresponding monomers or submitting the monomers to radiation, optionally in the presence of a photo-initiator.

In certain embodiments, the irradiation comprises ultraviolet electromagnetic radiation (wavelength about 10-400 nm), visible electromagnetic radiation (wavelength about 400-750 nm) or infrared electromagnetic radiation (radiation wavelength about 750-300,000 nm). In other embodiments, the electromagnetic radiation comprises ultraviolet or visible electromagnetic radiation.

Ultraviolet or UV light as described herein includes UVA light, which generally has wavelengths between about 320 and about 400 nm, UVB light, which generally has wavelengths between about 290 nm and about 320 nm, and UVC light, which generally has wavelengths between about 200 nm and about 290 nm. UV light may include UVA, UVB, or UVC light alone or in combination with other type of UV light. In certain embodiments, the UV light source emits light between about 350 nm and about 400 nm. In some embodiments, the UV light source emits light between about 400 nm and about 500 nm.

The photo-initiator contemplated within the invention is a molecule that, upon irradiation with a given wavelength at a given intensity for a given period of time, generates at least one species capable of catalyzing, triggering or inducing a polymerization or crosslinking. A photo-initiator known in the art may be employed, such as a benzoin ether and a phenone derivative such as benzophenone or diethoxyacetophenone.

Non-limiting examples of the photo-initiator contemplated within the invention are: 1-hydroxy-cyclohexyl-phenyl-ketone (IRGACURE® 184; Ciba, Hawthorne, NJ); a 1:1 mixture of 1-hydroxy-cyclohexyl-phenyl-ketone and benzophenone (IRGACURE® 500; Ciba, Hawthorne, N.J.); 2-hydroxy-2-methyl-1-phenyl-1-propanone (DAROCUR® 1173; Ciba, Hawthorne, N.J.); 2-hydroxy-1-[4-(2-hydroxyethoxy)phenyl]-2-methyl-l-propanone (IRGACURE® 2959; Ciba, Hawthorne, N.J.); methyl benzoylformate (DAROCUR® MBF; Ciba, Hawthorne, N.J.); oxy-phenyl-acetic acid 2-[2-oxo-2-phenyl-acetoxy-ethoxy]-ethyl ester; oxy-phenyl-acetic 2-[2-hydroxy-ethoxy]-ethyl ester; a mixture of oxy-phenyl-acetic acid 2-[2-oxo-2-phenyl-acetoxy-ethoxy]-ethyl ester and oxy-phenyl-acetic 2-[2-hydroxy-ethoxy]-ethyl ester (IRGACURE® 754; Ciba, Hawthorne, N.J.); alpha,alpha-dimethoxy-alpha-phenylacetophenone (IRGACURE® 651; Ciba, Hawthorne, N.J.); 2-benzyl-2-(dimethylamino)-1-[4-(4-morpholinyl)-phenyl]-1-butanone (IRGACURE® 369; Ciba, Hawthorne, N.J.); 2-methyl-1-[4-(methylthio)phenyl]-2-(4-morpholinyl)-1-propanone (IRGACURE® 907; Ciba, Hawthorne, N.J.); a 3:7 mixture of 2-benzyl-2-(dimethylamino)-1-[4-(4-morpholinyl) phenyl]-1-butanone and alpha,alpha-dimethoxy-alpha-phenylacetophenone per weight (IRGACURE® 1300; Ciba, Hawthorne, N.J.); diphenyl-(2,4,6-trimethylbenzoyl) phosphine oxide (DAROCUR® TPO; Ciba, Hawthorne, N.J.); a 1:1 mixture of diphenyl-(2,4,6-trimethylbenzoyl)-phosphine oxide and 2-hydroxy-2-methyl-1-phenyl-1-propanone (DAROCUR® 4265; Ciba, Hawthorne, N.J.); phenyl bis(2,4,6-trimethyl benzoyl) phosphine oxide, which may be used in pure form (IRGACURE® 819; Ciba, Hawthorne, N.J.) or dispersed in water (45% active, IRGACURE® 819DW; Ciba, Hawthorne, N.J.); a 2:8 mixture of phosphine oxide, phenyl bis(2,4,6-trimethyl benzoyl) and 2-hydroxy-2-methyl-1-phenyl-1-propanone (IRGACURE® 2022; Ciba, Hawthorne, N.J.); IRGACURE® 2100, which comprises phenyl-bis(2,4,6-trimethylbenzoyl)-phosphine oxide); bis-(eta 5-2,4-cyclopentadien-1-yl)-bis-[2,6-difluoro-3-(1H-pyrrol-1-yl) phenyl]-titanium (IRGACURE® 784; Ciba, Hawthorne, N.J.); (4-methylphenyl) [4-(2-methylpropyl) phenyl]-iodonium hexafluorophosphate (IRGACURE® 250; Ciba, Hawthorne, N.J.); 2-(4-methylbenzyl)-2-(dimethylamino)-1-(4-morpholinophenyl)-butan-1-one (IRGACURE® 379; Ciba, Hawthorne, N.J.); 4-(2-hydroxyethoxy)phenyl-(2-hydroxy-2-propyl)ketone (IRGACURE® 2959; Ciba, Hawthorne, N.J.); bis-(2,6-dimethoxybenzoyl)-2,4,4-trimethylpentylphosphine oxide ; a mixture of bis-(2,6-dimethoxybenzoyl)-2,4,4-trimethylpentylphosphine oxide and 2-hydroxy-2-methyl-1-phenyl-propanone (IRGACURE® 1700; Ciba, Hawthorne, N.J.); titanium dioxide; and mixtures thereof.

In certain embodiments, one or more accelerators are utilized in the photopolymerization. Amine accelerators can be used as polymerization accelerators, as well as other accelerators. Polymerization accelerators suitable for use are various organic tertiary amines well known in the art. In visible light curable compositions, the tertiary amines are generally acrylate derivatives such as dimethylaminoethyl methacrylate and, particularly, diethylaminoethyl methacrylate (DEAEMA), EDAB and the like, in an amount of about 0.05 to about 0.5 wt. %. The tertiary amines are generally aromatic tertiary amines, such as tertiary aromatic amines such as EDAB, 2[4-(dimethylamino)phenyl]ethanol, N, N-dimethyl-p-toluidine (commonly abbreviated DMPT), bis(hydroxyethyl)-p-toluidine, triethanolamine, and the like.

The invention includes a pharmaceutical composition comprising at least one material of the invention and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Administration/Dosing

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

### Pharmaceutical Compositions

The present invention provides a pharmaceutical composition comprising at least one material of the present invention and a pharmaceutically acceptable carrier. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the present invention include anti-inflammatories, including corticosteroids, and immunosuppressants, chemotherapeutic agents, antibiotics, antivirals, antifungals, and the like.

Controlled- or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, and caplets, which are adapted for controlled-release are encompassed by the present invention.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In a preferred embodiment of the invention, the compounds of the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present invention are not limited to the particular formulations and compositions that are described herein.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

### Kits

The invention also provides kits including a material and/or a composition of the invention, and optionally another therapeutic agent, as described herein elsewhere, and instructions for its use. The instructions will generally include information about the use of the compositions in the kit for the treating, ameliorating, and/or preventing the diseases and disorders contemplated here. The instructions may be printed directly on a container inside the kit (when present), or as a label applied to the container, or as a separate sheet, pamphlet, card, or folder supplied in or with the container.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

Methods and materials

Hexamethyldisilazane (HDMS, CAS 999-97-3), poly(ethylene glycol)-block-polypropylene glycol)-block-poly(ethylene glycol) (Pluronic 123, CAS 9003-11-6), tetraethyl orthosilicate (TEOS, CAS 78-10-4), poly(vinyl alcohol) (PVA) (CAS 9002-89-5), poly(acrylic acid) (PAA) (CAS 9003-01-4), toluene (CAS 108-88-3), hydrochloric acid (37%, CAS 7647-01-0), 3-(4,5-dimethylthiazol-2-yl)-2,5-diphenyltetrazolium bromide (MTT), and dimethyl sulfoxide (DMSO, CAS 67-68-5) were purchased from Sigma-Aldrich. N-Trimethoxysilylpropyl-N,N,N-trimethylammonium chloride (C₉H₂₄ClNO₃Si, abbreviated as TOSPTA, MW=257.83, 50% in methanol; CAS 35141-36-7) was purchased from Gelest, Inc. (Morrisville, Pa, USA). Dexamethasone (abbreviated as DEX, 98%, MW=392.47, and CAS 50-02-2) was purchased from Alfa Aesar. Ethanol (CAS 64-17-5) was obtained from Decon Labs, Inc. Diethyl ether (CAS 60-29-7) and sodium hydroxide (CAS 1310-73-2) were purchased from Fisher Scientific. Phosphate buffered saline (PBS, pH 7.4) solution, Fetal bovine serum (FBS), and Dulbecco's modified eagle's medium (DMEM) were purchased from Gibco-Invitrogen. All the reagents were used as received without further purification.

Synthesis of SBA-15 Mesoporous Silica Nanoparticles and their Surface Functionalization

First Pluronic 123 (4 g) was dissolved in a solution containing concentrated HCl (37%) (24 mL) and distilled water (104 mL). After adjusting the temperature of the solution at 45° C., TEOS (8.5 g) was added into the solution. The solution was then vigorously stirred for 24 h, and after which, it was kept in oven at 80° C. for 24 h. The as-prepared mesostructured SBA-15 nanoparticles were recovered by filtration, washed copiously with distilled water, and then dried in oven at 40° C. Prior to template extraction, the external surface of the as-prepared mesostructured SBA-15 nanoparticles were modified with quaternary ammonium groups by stirring 300 mg of the sample in TOSPTA/toluene (1 mL/150 mL) solution for 6 h at 80° C. The mesostructured SBA-15 nanoparticles whose external surfaces were capped with quaternary ammonium groups were stirred in a solution (100 mL) of diethyl ether:ethanol (1:1 ratio) for 24 h to remove the Pluronic 123 template and to create the channel pores. The resulting mesoporous SBA-15 nanoparticles were then stirred in a solution of hexamethyldisilazane (HMDS) (1.5 mL) in either anhydrous toluene or 2-propanol (30 mL) for 2 h at 60° C. to graft methyl groups onto their channel walls. The solid products were recovered by filtration, washed with copious amount of ethanol, and dried in oven at 40° C. The resulting two different SBA-15 mesoporous silica nanoparticles whose external surfaces were capped with quaternary ammonium groups and whose internal surfaces were modified with higher or lower density of methyl groups were labeled as SPH-4 and SPH-4L, respectively.

### Fabrication of Aerogels

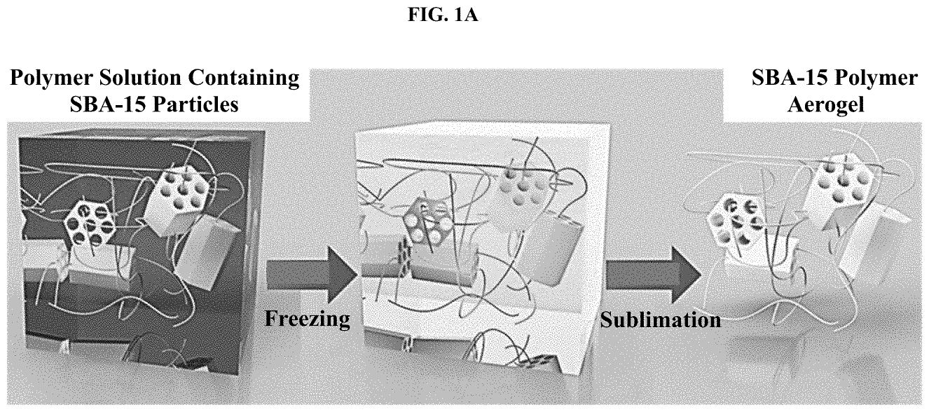

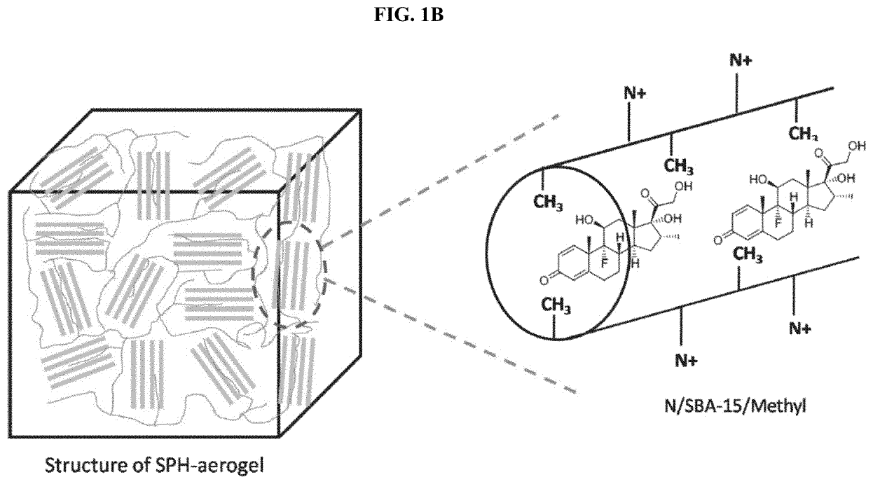

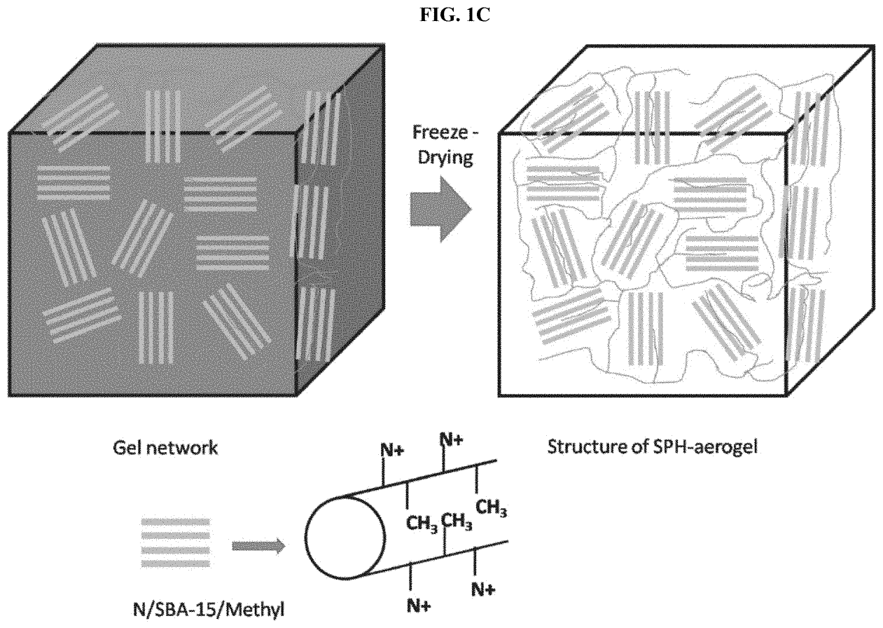

Mesoporous SBA-15 nanoparticles were synthesized as reported in the literature (Xie, et al., 2008, J. Phys. Chem. C 112:9996-10003; Silva & Asefa, 2012, Adv. Mat. 24:1878-1883). Their external surfaces were then modified with quaternary ammonium groups by grafting TOSPTA on them, and their internal walls were modified with methyl groups by grafting HMDS on them. The resulting co-functionalized mesoporous silica nanoparticles, with quaternary ammonium groups on their external surfaces and methyl groups on their internal pore surfaces, were mixed with PVA, PAA, and water in different ratios, as described in Table 1, to form stable dispersions. The dispersions were immediately poured into a plastic container containing multiple cylindrical wells, with ca. 6×7.5 mm in length x diameter, which served as hard templates to prepare beads of aerogels. The samples were then frozen, lyophilized, removed from the plastic template, and placed in an oven for 45 min at 160° C. to promote the cross-linking reaction between PVA and PAA. A schematic diagram of the fabrication procedure of the aerogels is shown in FIG. 1A. The resulting aerogels comprising functionalized SBA-15 mesoporous silica nanoparticles with cross-linked PVA and PAA were denoted as “Silica-Polymer Hybrid (SPH)”, followed by a number to represent the specific condition used for the synthesis of the particular material listed in Table 1; for instance, “SPH-1” aerogel was synthesized by using the precursors mentioned under “condition 1” in Table 1. The structures and compositions of the aerogel beads were then characterized by various analytical methods, and their drug release properties in vitro were studied.

TABLE 1
Ratio of the reagents used to synthesize different SPH aerogels.
Condition

| | | | Mesoporous | | | |
|---|---|---|---|---|---|---|
| | | | Silica | | PVA-to- | |
| | PVA | PAA | Nanoparticles | H₂O | PAA | S/L Ratio |
| Samples | (mg) | (mg) | (mg) | (mL) | Ratio | (mg/mL) ^{a)} |
| SPH-1 | 100 | 100 | 50 | 2 | 1 | 125 |
| SPH-2 | 200 | 200 | 100 | 2 | 1 | 250 |
| SPH-3 | 150 | 50 | 50 | 2 | 3 | 125 |
| SPH-4 | 300 | 100 | 100 | 2 | 3 | 250 |
| SPH-5 | 50 | 50 | 25 | 2 | 1 | 67.5 |
| SPH-6 | 400 | 400 | 200 | 2 | 1 | 500 |

^{a)} S/L: Solid-to-liquid ratio used to prepare the precursor gels.

### Characterizations

The composition of the aerogels was characterized by obtaining the Fourier transform infrared (FTIR) spectra of the samples with a FTIR spectrometer (Brüker, Model Vertex-70/70v). The spectra were acquired by running 128 scans from 500 to 4,000 cm⁻¹ with a resolution of 4 cm⁻¹ using samples prepared on KBr discs containing 1.0 wt. % of each SPH sample. The surface morphology of the materials was examined with a Zeiss Sigma field emission scanning electron microscope (SEM) using samples that were coated with a thin layer of sputtered gold. The bulk density of the samples was determined by dividing the average weights of the beads (measured using an analytical balance) by the volumes of the beads (obtained from the volumes of the plastic templates used to prepare the aerogels).

The formation of cross-linkages in the polymers was characterized using Fourier transform infrared (FTIR) spectroscopy. To differentiate the carboxylic acid groups from the esters groups formed by the solid-state reaction, which can have overlapping peaks on FTIR spectra, the aerogels were treated with a basic solution to convert the remaining carboxylic acid into carboxylate groups by putting the SPH aerogels in a flask containing 30 mL of aqueous 0.1 M NaOH solution at room temperature for 30 min and then taking them out of the solution and letting them dry under ambient condition.

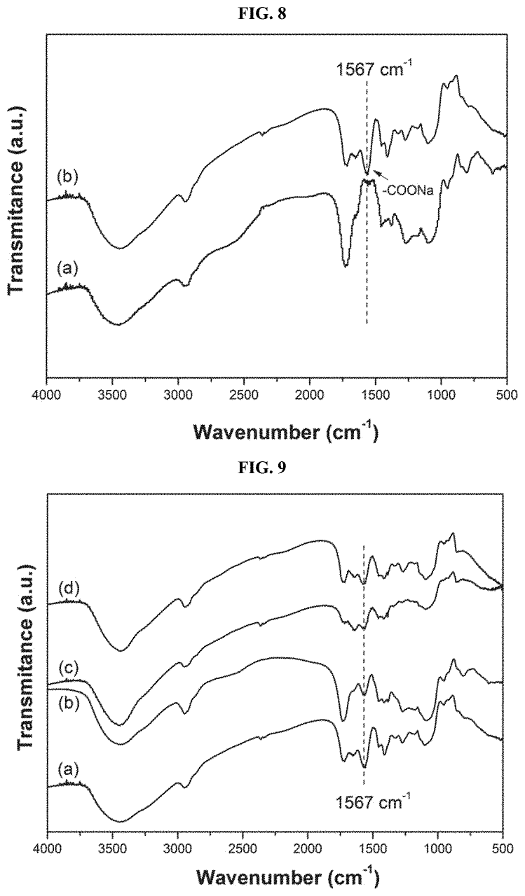

The FTIR spectra of the SPH-1 before and after treatment with NaOH solution are presented in FIG. 8. The spectra show some features that are common in all the cases (FIG. 9). For instance, a broad band at ca. 3480 cm⁻¹ due to O—H stretching, a band at ca. 2,940 cm⁻¹ attributable to the C-H stretching vibration of CH₂ groups from both polymers, and the band at 1,240 cm⁻¹ due to the O—H bending vibration were observed. However, the region between 1,750 to 1,500 cm⁻¹, where the C═O stretching bands typically appear and which is important to confirm formation of cross-linked bonds between PVA and PAA through a solid-state reaction, was particularly monitored. In the spectra of samples before being subjected to treatment with basic solution, a single peak at 1,720 cm⁻¹ associated with carbonyl groups was observed. In the spectra of the sample after treatment with a dilute basic solution though, it was possible to see the change in the peaks in the carbonyl region or the appearance of an intense band at 1,567 cm⁻¹ that is attributed to C═O stretching of carboxylate groups. More importantly, after the treatment with a base, the peak at 1,720 cm⁻¹ was not completely vanished. Consequently, the remaining peak at 1,720 cm⁻¹, after the samples are kept in 0.1 mol/L NaOH solution for 30 min, was due to the carbonyl moiety of ester groups formed in the cross-linking reaction.

The water uptake and stability of the beads were determined by dipping the beads in 0.1 mol/L PBS solution at pH 7.4 at room temperature. The beads were removed from the solution at regular intervals of time, laid on a paper-towel to remove excess water, and then weighed. The water uptake by the beads (I) was determined using Equation (1):

$$I ( \% ) = \frac{{W}_{i}}{{W}_{s}} \times 100 \qquad Equation ( 1 )$$

where Wᵢ is the weight of the swollen SPH beads and Wₛ is the weight of the initial dried SPH beads. All determinations were carried out in triplicate. The samples synthesized under different conditions were kept in PBS solution for one month to assess their structural stability.

N₂ adsorption-desorption data for the materials were obtained with Micromeritics Tristar-3000 instrument. Based on the data, the surface area of the aerogels and the functionalized SBA-15 mesoporous silica nanoparticles were obtained by the Brunauer-Emmett-Teller (BET) method and their pore size distributions were determined by the Barrett-Joyner-Halenda (BJH) method. The materials were characterized by thermogravimetric analysis using a PerkinElmer TGA7 instrument, by heating samples at 10° C./min under a flow of air at a rate of 20 mL/min.

Loading of Dexamethasone (DEX) into SPH Aerogels

The SPH beads were loaded with DEX by dipping 100 mg of each sample into ethanol/water (1:4 v/v) solution containing DEX (0.6 mg in 15 mL of solution), and then stirring the mixture with a magnetic stirrer for 24 h at room temperature. After removing the DEX-loaded SPH beads with a pair of tweezers, the UV-Vis spectra of the supernatants were analyzed with a UV-Vis spectrometer (Lambda 850, PerkinElmer). By monitoring the peak at 242 nm corresponding to absorption maximum of DEX, the amount of DEX in the solutions, and then in the beads, were quantified. Prior to this, an analytical calibration curve (with R²=0.999) was obtained using different known concentration of DEX solutions ranging from 0.10 to 10.0 mg/L. Based on the calibration curve and the measured absorption bands of the supernatants, the encapsulation efficiency of the beads for DEX was determined using Equation (2):

$$\text{Encapsulation Efficiency} ( \% ) = \frac{{[ DEX ]}_{i} - {[ DEX ]}_{s}}{{[ DEX ]}_{i}} \times 100 \qquad Equation ( 2 )$$

where [DEX]ᵢ is the initial concentration of DEX and [DEX]ₛ is the residual amounts of DEX in the supernatants after adsorption.

### Drug Adsorption Isotherms

The adsorption process for DEX in the aerogels was monitored by placing 50 mg of SPH beads into a sealed flask containing solutions of DEX with different concentrations ranging from 0.05 to 1.8 mg/mL and stirring the mixtures with a magnetic stirrer for 24 h at 25° C. The amount of DEX adsorbed into the beads was determined by using UV-Vis spectroscopy.

### Drug Release Studies

In vitro drug release experiments were performed using DEX-loaded SPH beads in 0.1 mol/L PBS at pH 7.4. For each experiment, 100 mg of sample was kept in a sealed flask containing 40 mL of PBS solution under gentle mechanical stirring at 37° C. In regular intervals of time, an aliquot (2 mL) of each sample was then removed from the flask and their absorbance at 242 nm was monitored. The fractions of DEX released from the beads into their corresponding solutions were then calculated using Equation (3):

$$Equation ( 3 )$$

$$Fraction of DEX Released ( \% ) = \frac{amount released}{amount encapsulated} \times 100$$

### Cytotoxicity Tests of the Aerogels

The biocompatibility/cytotoxicity of the aerogels was evaluated using African green monkey kidney cells (Vero cells—ATCC® CCL81) and MTT assay (Follmann, et al., 2016, J. Colloid and Interface Sci. 474:9-17). Vero cells were maintained in DMEM, supplemented with 10% of fetal bovine serum (FBS) and incubated under normal cell culture condition (at 37° C. in 5% CO₂ atmosphere under controlled humidity) for 96 h. Typically, cells were first seeded in 96-well plate at the density 2.5×10⁵ cells per well after trypsinization, and cultured in a humidified incubator at 37° C. under 5% CO₂ atmosphere. After letting the cells adhere for 24 h, the aerogel materials and functionalized-silica samples (at three different concentrations: 50, 100 and 200 μg/mL) were dispersed over the cells, and the plates were incubated under the same condition as described elsewhere herein. Cell viability was determined after 72 h with MTT assay. Briefly, the culture medium was removed and replaced with 500 μL of MTT solution (2.0 mg/mL in DMEM). The samples were incubated for 4 h at 37° C. to help the cells take up MTT and to let the formation of formazan crystals. The culture media were then removed, and the crystals were solubilized in DMSO (1 mL). Their absorbance was measured at 570 nm by using a microplate spectrophotometer (Bio Tek-Power Wave XS). The toxicity/biocompatibility of the materials was estimated by comparing the results for the materials with respect to those for the untreated cells (control experiments). For each sample, the measurement and data analyses were performed three times.

### Cell Growth Assay in the Presence of Aerogels

In addition to the cytotoxicity assay, cell proliferation was evaluated against NCTC clone 929 [L cell, L-929, derivative of Strain L] (ATCC®CCL-1™) fibroblast cells using MTT assay. The cells were obtained from Mus musculus mouse, specifically from subcutaneous connective tissue and areolar and adipose, and they had fibroblast morphology. The L929 cells were cultured in DMEM, supplemented with 10% of FBS and kept inside an incubator at 37° C. in 5% CO₂ atmosphere. After 90% of confluence, the cells were washed with PBS, trypsinized, and resuspended in DMEM. All the samples were placed in 96-well cell culture plates, and the cells (2.5×10⁵ cells/mL) were then seeded on the top of the samples. After placing the plates containing the samples inside the humidified incubator at 37° C. in 5% CO₂ atmosphere for 72 h, the culture medium was removed and replaced with MTT solution (2.0 mg/mL in DMEM). The absorbance of the culture medium at 570 nm was then determined under the same condition as described above. The cell proliferation assays were performed three times for each sample.

### Antibacterial Susceptibility Testing

The minimum inhibitory concentration (MIC) and minimum bactericidal concentration (MBC) results of nanoparticles are summarized in Table 2. The in vitro results were classified according to the MIC value described by Brambilla et al., 2017, Revista Brasileira de Farmacognosia 27:112-117. For values smaller than 100 μg/mL, the antibacterial activity was considered good; for values between 100 and 500 μg/mL the activity was considered moderate; for values between 500 to 1000 μg/mL the activity was considered weak; and for values larger than 1000 μg/mL the nanoparticles did not present activity.

Based on the results in Table 2, the functionalized silica displayed bactericidal activity against all the strains studied, with better results especially against S. aureus (gram positive) and B. subtilis (gram positive) compared with E. coli (gram negative) and P. aeruginosa (gram negative) bacteria. The pure silica did not show antibacterial activity. These results are expected since the external walls of the functionalized SBA-15 mesoporous silica particles were intentionally functionalized with quaternary ammonium groups, which have bactericidal activity. These groups exhibit bactericide activity due to their permanent positive charges, which interacts strongly with the negative charges on bacterial membranes, causing membrane rupture. The different values found to the gram positive and gram-negative bacteria could be attributed to the outer membrane barrier, existent only in gram-negative bacteria.

TABLE 2
Minimal inhibitory concentration (MIC) and minimal bactericidal
concentration (MBC) of pure silica and functionalized silica.

| | MIC (μg/ml) | MCB (μg/ml) | MIC (μg/ml) | MCB (μg/ml) |
|---|---|---|---|---|
| Strains | Functionalized Silica | | Pure Silica | |
| E. coli | 250.0 | 500.0 | >1000.0 | N/E* |
| S. aureus | 62.5 | 125.0 | >1000.0 | N/E |
| B. subtilis | 125.0 | 125.0 | >1000.0 | N/E |
| P. aeruginosa | 250.0 | 250.0 | >1000.0 | N/E |

*Not evaluated

The minimal inhibitory concentration (MIC) of the nanoparticles (pure silica and organoc-modified silica) was evaluated against Escherichia coli (E. coli—ATCC 25922), Staphylococcus aureus (S. aureus—ATCC 25923), Bacillus subtilis (B. subtilis—ATCC 6623), and Pseudomonas aeruginosa (P. aeruginosa—ATCC 27853) strains by microdilution techniques in Mueller-Hilton broth (Merck), as described by Toledo et al., 2011, J. Ethnopharmacology 133:420-425). Inoculates were prepared in the same medium at a density adjusted to a 0.5 McFarland turbidity standard (10⁸ colony-forming units (CFU)/ml) and diluted 1:10 for the broth microdilution method. Microtiter trays were incubated at 37° C. and the values of MIC were determined after the incubation period of 24 h. MIC is defined as the lowest concentration of material that results in no visible growth in the strain. The minimum bactericidal concentration (MBC) was performed in Mueller-Hinton agar after 24 h in an incubation temperature of 37° C. MBC is defined as the lowest concentration that yields negative subcultures or only one colony.

### Example 1

An innovative synthetic methodology involving freeze-drying and solid-state reaction was used to produce new and appealing inorganic/organic or mesoporous silica/polymer hybrid aerogel beads for sustained drug delivery. The materials, which are referred to hereafter also as SPH materials, comprise mesoporous silica nanoparticles and biocompatible polymers, PAA and PVA (FIGS. 1A-1C). By varying the relative ratios of the mesoporous silica nanoparticles, PVA and PAA, as well as the condition used to make the materials, as compiled in Table 1, aerogels with different structures and appealing properties for drug delivery, were prepared.

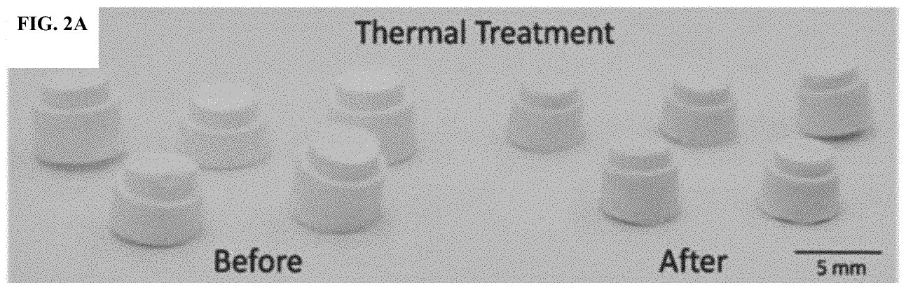

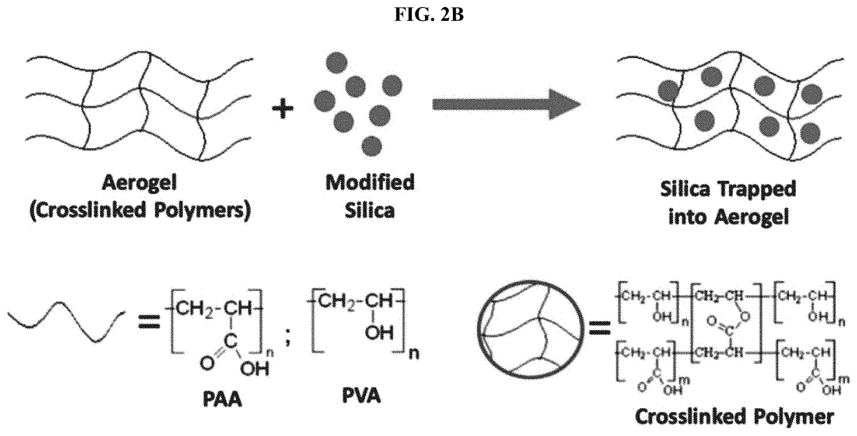

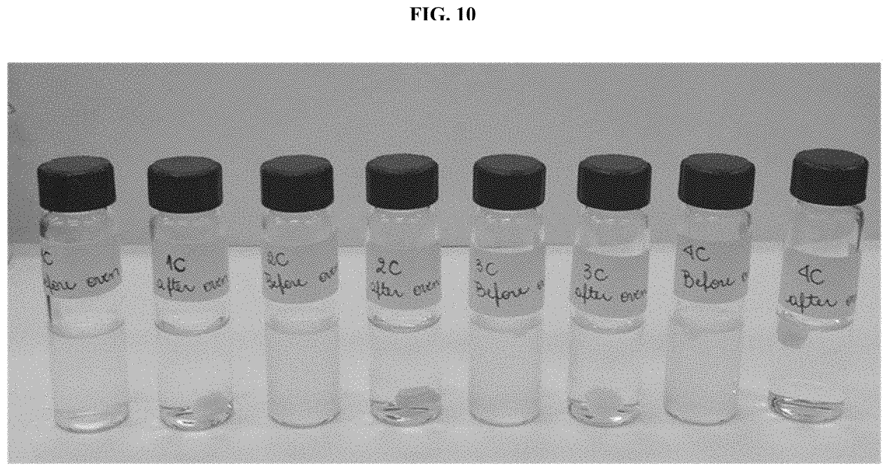

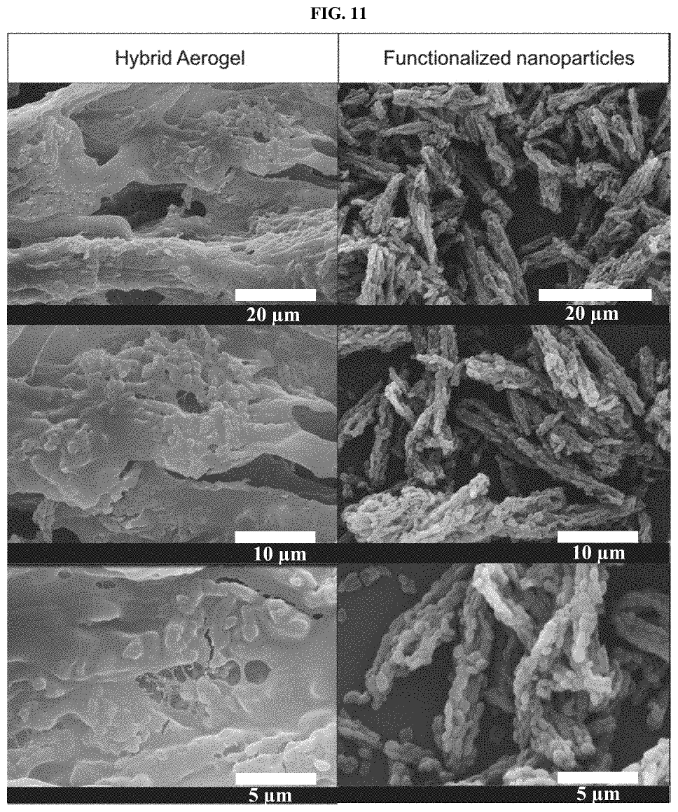

The digital images of the SPH aerogel beads (FIGS. 2A-2B) showed that their sizes and shapes mimicked those of the underlying plastic templates used to make them. Without wishing to be limited by any theory, this may be because the removal of water by freeze-drying did not change the original bulk volume of the beads. The occurrence of a solid-state reaction crosslinking PAA and PVA was confirmed by FTIR spectroscopy (see FIGS. 8-9). It was also qualitatively evident from the observed change in the color of the samples from white to yellowish (FIGS. 2A-2B). Subsequent thermal treatment of the beads made them more stable, allowing them to maintain their structural integrity even when immersed in aqueous basic media (FIG. 10) as well as in neutral solutions. In contrast to this, the beads before thermal treatment were susceptible to structural deformation when dipped into aqueous basic solutions. As shown in the schematic illustration in FIG. 2Bb, the hybrid material is formed by covalent bonds formed between the two polymers, or the condensation reaction between the —OH groups in PVA and the —COOH groups in PAA; so, most of the polymer chains are in neutral form near neutral pH.

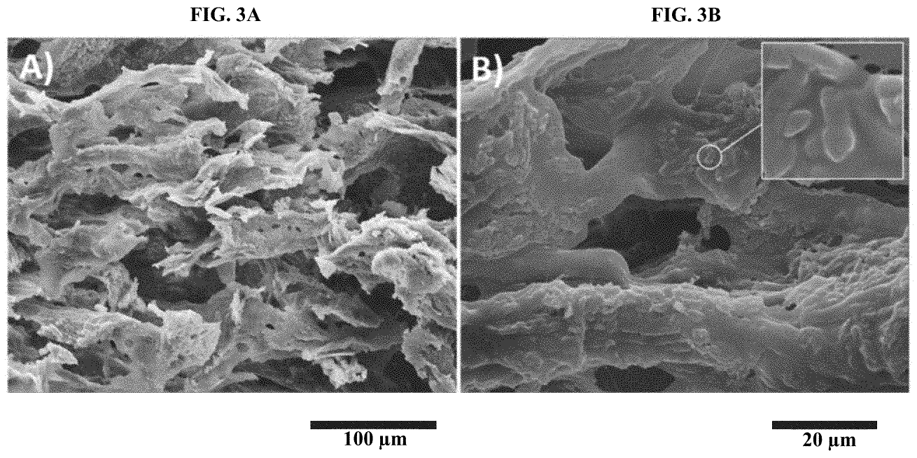

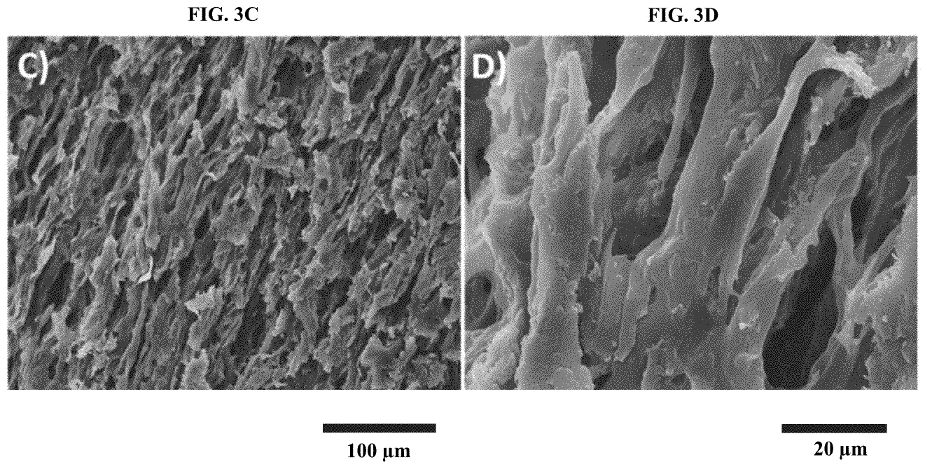

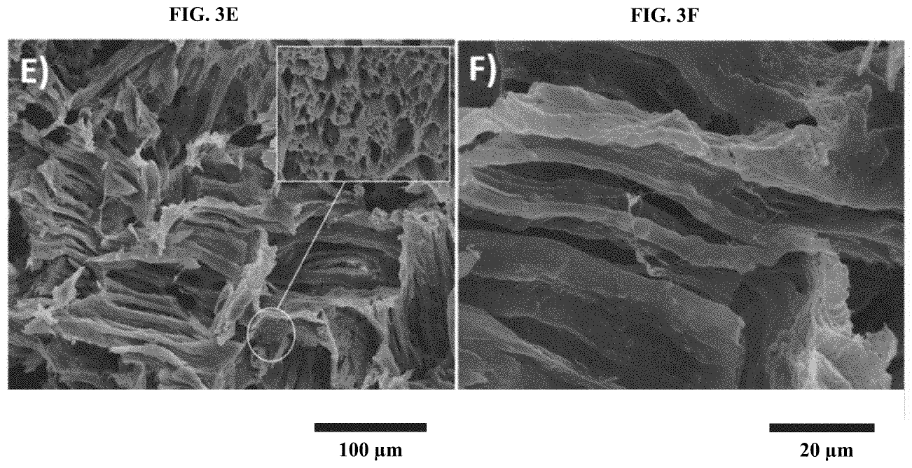

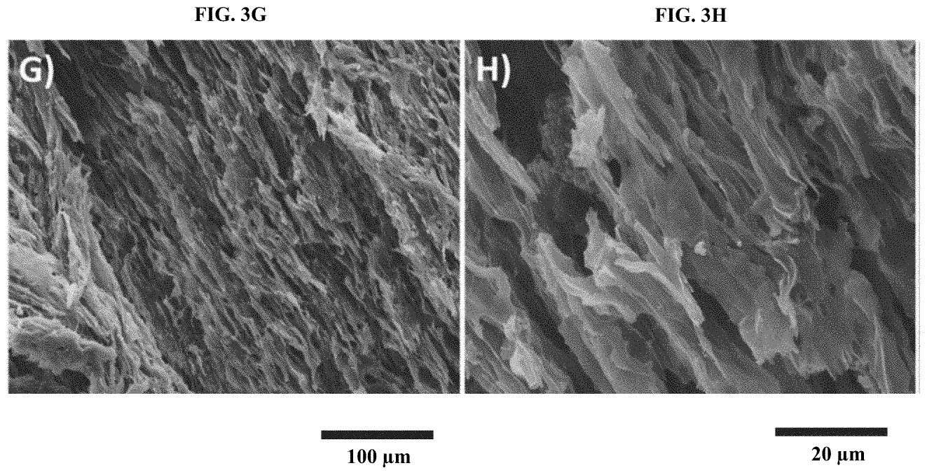

The structures of SPH aerogels were analyzed using SEM (FIGS. 3A-3H). Based on the SEM images, the materials obtained after removal of water by sublimation appeared to have highly porous and uniform structures, although the sizes of their pores and structures generally varied depending on the conditions used to make them. For instance, SPH-1 had larger pores, as high as ca. 80 μm, without any evident pore alignment, whereas SPH-2, SPH-3, and SPH-4 had smaller pores with better pore alignment. In particular, the pores in SPH-2 and SPH-4 appeared one-dimensionally aligned, more so than those in SPH-1 and SPH-3. The alignment of the pores particularly in some of the aerogels (i.e., SPH-2 and SPH-4) was most likely the result of organized crystallization of water (from the solution) within the structures of the materials. As the freezing process proceeded, ice crystals could grow in an ordered manner as segregated phases. When these ice crystals were removed via sublimation, the well-aligned pores could form.

The relative amount of water in the precursors was a major factor determining to what extent aligned pores form in the aerogels. In other words, when the amount of water in the solution was relatively less or the solid-to-liquid (S/L) ratio of the solution was relatively high, as in the precursors used to make SPH-2 and SPH-4, the ice crystals could grow in more controlled and aligned manner. On the other hand, as the amount of water in the solution was relatively high or the S/L ratio was relatively small, only bigger and more disorganized ice crystals could grow and more disordered pores could ultimately form in the aerogels. Without wishing to be limited by any theory, besides the relative amount of water in the precursors, the PVA-to-PAA ratio can have also dictated how the pores in the aerogels formed. This was because, for the same S/L ratio, the materials prepared from the precursors containing higher PAA-to-PVA ratios were found to have better aligned pore structures. This could be appreciated especially when comparing the pore structures of SPH-1 with those of SPH-3, or the pore structures of SPH-2 with those of SPH-4.

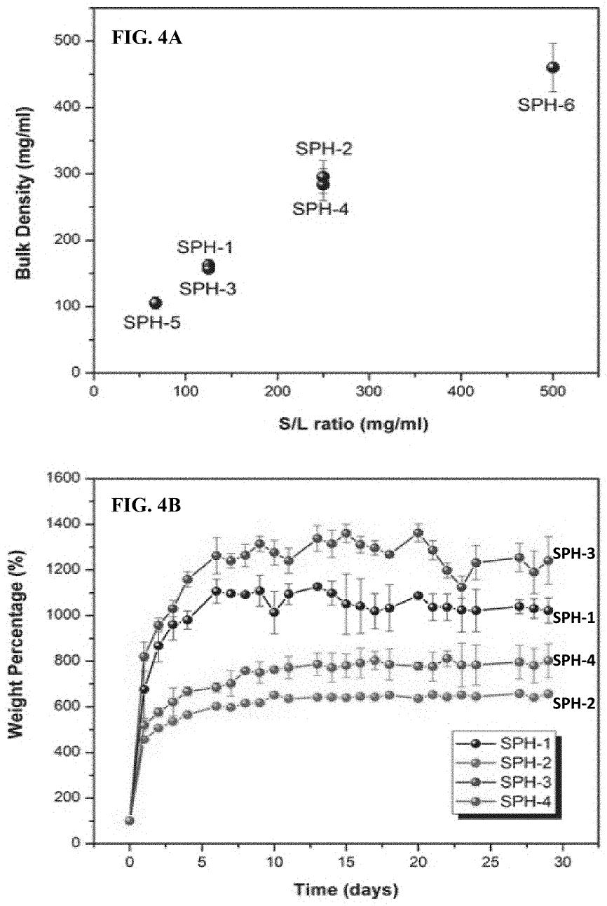

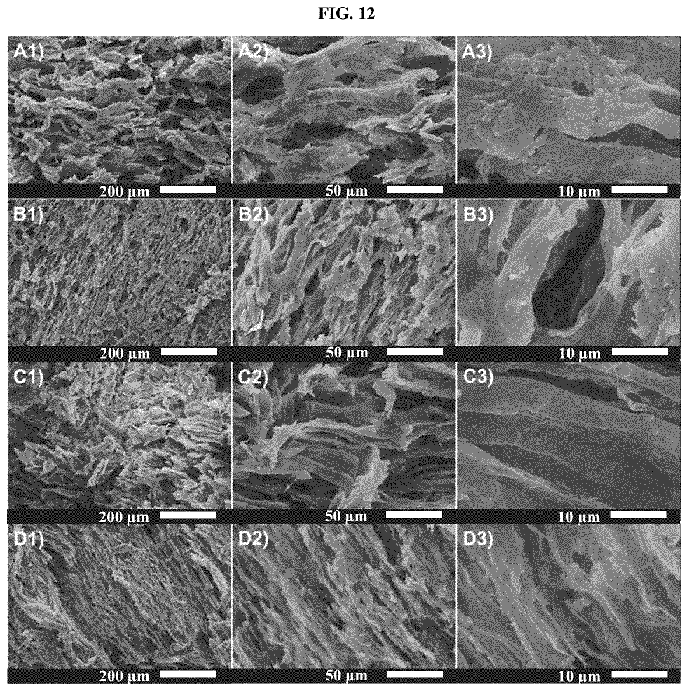

In the SEM images of all the hybrid aerogel materials (FIGS. 3A-3H), no isolated or segregated mesoporous silica nanoparticles were seen. This indicated that the mesoporous silica nanoparticles were completely dispersed/incorporated within the polymer matrices. Furthermore, upon more careful inspection of the SEM images, it was possible to see contours of some mesoporous silica nanoparticles that were partly exposed from the polymer matrices. SEM images at different magnifications of the functionalized mesoporous silica nanoparticles and SPH-1 material were obtained and compared side-by-side in FIGS. 4A-4B. The image of SPH-1 showed some polymer-trapped mesoporous silica nanoparticles, whose shapes and sizes matched with those seen in the images obtained for the pure functionalized mesoporous silica nanoparticles. So, once again, it can be said that the mesoporous silica nanoparticles must have been fully integrated into and tightly held within the hyperbranched polymer networks of the aerogel materials. Additional SEM images of the aerogel materials are shown in FIG. 12.

The bulk density of all the aerogel materials varied almost linearly with the S/L ratio of the precursors/gels used to synthesize materials (FIG. 4A). The density of the aerogel beads was dependent on the free pore volume present inside the beads. Although SPH-1 and SPH-3 were made from precursors with the same S/L ratio, they showed slightly different densities. The same could be said about SPH-2 and SPH-4. The slight differences in the densities between the two sets of materials must have been the result of the differences in the total mass of the materials, which could vary depending on how much water molecules were lost as a by-product when the polymers underwent cross-linking through esterification reaction. The aerogels prepared from higher ratios of PVA-to-PAA (e.g., 3:1) had lower densities, as their corresponding cross-linked polymers were more branched or not highly condensed. The opposite was true for those aerogels prepared from precursors with relatively lower ratios of PVA-to-PAA (e.g., 1:1).

As can be seen in FIG. 4B, the aerogels swelled in PBS solution, with the swelling reaching equilibrium within ca. 6 days. The aerogel materials possessing low bulk density, such as SPH-1 and SPH-3, exhibited the highest water uptake; e.g., SPH-3 displayed a whopping 1300% higher weight as compared with its initial weight. When comparing the amount of water taken up by different aerogels possessing similar densities with one another (i.e., SPH-1 versus SPH-3 and SPH-2 versus SPH-4), a higher water uptake was exhibited by the aerogels synthesized from precursors containing higher PVA-to-PAA ratios (SPH-3 and SPH-4). This is because these aerogels had more leftover PVAs, which could not react with PAA; as a result, the aerogels had more free OH groups and higher hydrophilicity to accommodate more water molecules.

TABLE 3
Encapsulation efficiency (EE) of dexamethasone (DEX) in the aerogels.

| | | DEX/SPH |
|---|---|---|
| Samples | EE (%) | (mg/g) |
| SPH-1 | 76.1 ± 0.3 | 6.0 ± 1.6 |
| SPH-2 | 78.0 ± 1.5 | 5.0 ± 0.6 |
| SPH-3 | 76.0 ± 2.7 | 5.1 ± 0.8 |
| SPH-4 | 79.0 ± 1.4 | 6.0 ± 1.2 |
| SPH-4P^{a)} | 76.0 ± 0.1 | 5.1 ± 0.1 |

^{a)}This sample is similar to SPH-4 except that it was prepared using 2-propanol as a solvent during the grafting of HMDS on the internal pore walls of the mesoporous silica nanoparticles.

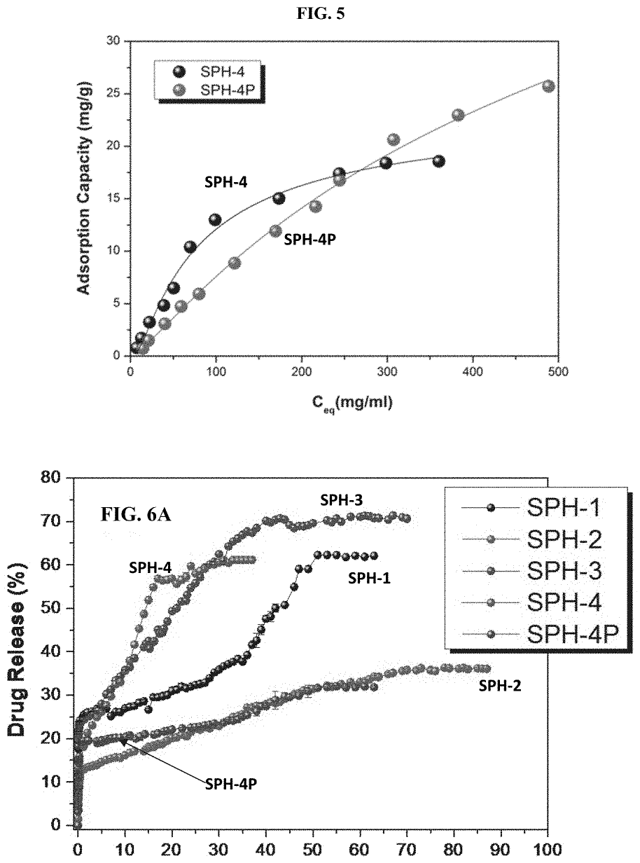

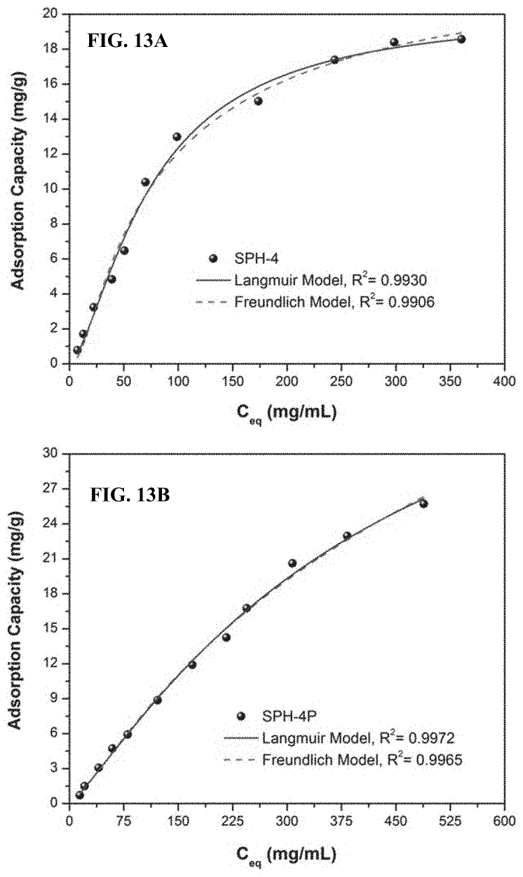

The encapsulation efficiency (EE) of the aerogels for DEX was found to be high (ca. 75% based on the original concentration of DEX present in the solution), as shown in Table 3. The high values of EE of these hydrogels for this hydrophobic drug can be mainly due to the methyl groups grafted on the inner walls of the mesoporous silica nanoparticles present in the hydrogels. The importance of the grafted methyl groups in helping the hydrogels to adsorb DEX was further evaluated by synthesizing another aerogel material with similar structure and composition as those in SPH-4, but with less methyl groups; this was done by using 2-propanol (instead of toluene) as a solvent to graft HMDS onto the surfaces of the mesoporous silica nanoparticles. Note that, compared with toluene, the most commonly used solvent for grafting organic groups from organoalkoxysilanes onto silica surfaces, 2-propanol is not effective in doing so. Hence, the density of grafted methyl groups onto the pore surfaces of the mesoporous silica nanoparticles is less when the latter solvent is used. This makes the aerogel resulting from it, referred to as SPH-4P, to unsurprisingly have weaker affinity to adsorb DEX molecules. This was appreciated when comparing the adsorption profile of SPH-4 for DEX versus that of SPH-4P (FIG. 5). Compared with SPH-4, SPH-4P exhibited a weaker adsorption affinity for DEX, as seen by its lower adsorption capacity for DEX in Ceq ranging from 0 to around 250 mg/mL. The adsorption isotherms of all the materials could be fitted well using either the Langmuir or the Freundlich model, as both of them gave very similar R² values (FIGS. 13A-13B). The adsorption properties of the aerogels analyzed based on the Freundlich model are presented in FIG. 5.

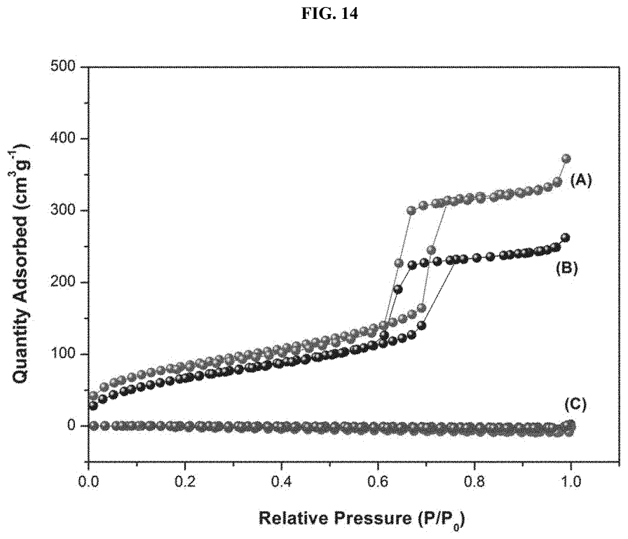

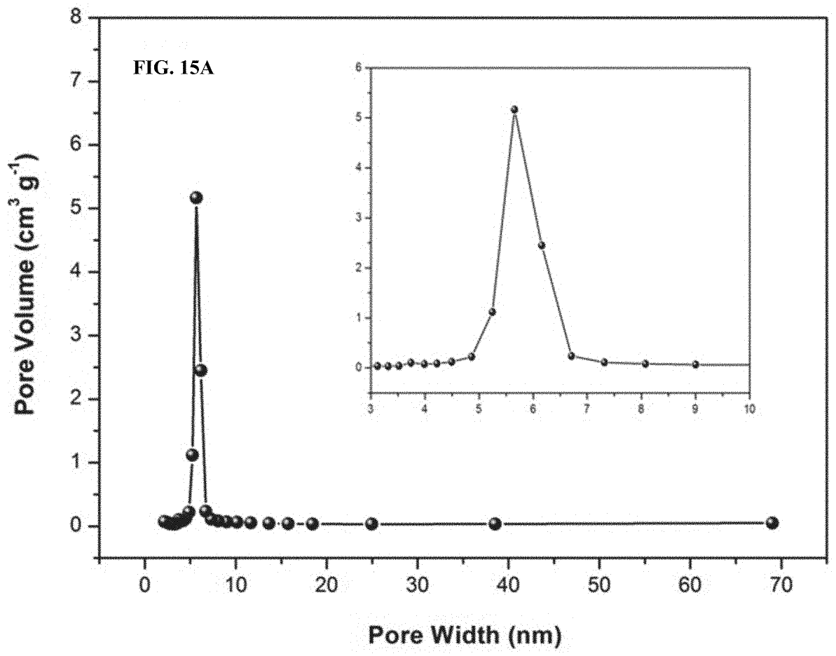

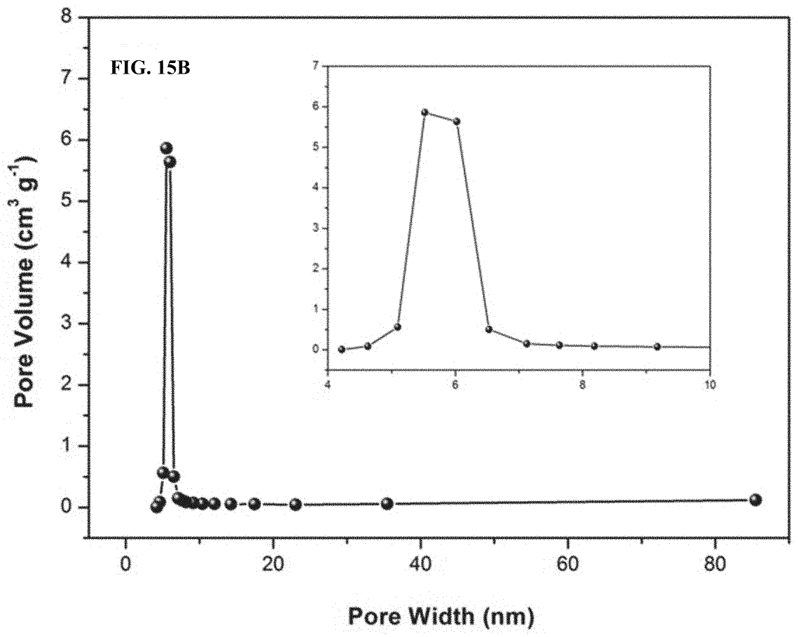

According to the results depicted in FIG. 5, unlike SPH-4, SPH-4P was not saturated by the drug when incubated in DEX solutions in the concentration ranges and at the incubation times that the adsorption experiments were conducted. On the other hand, SPH-4 got saturated when it was kept in DEX solution with a concentration of 300 mg/mL, showing an adsorption capacity of ca. 18.5 mg/g. However, SPH-4P did not become saturated even when it was kept in a solution of DEX with almost twice as much concentration. Nevertheless, even though SPH-4P showed less adsorption affinity towards DEX at low concentrations of DEX than did SPH-4, the former was able to accommodate a higher amount of DEX or show a higher adsorption capacity for DEX. This was in line with the results obtained with N₂ porosimetry for these two materials (FIGS. 8 and 14). The BET surface area of the methyl-grafted mesoporous silica nanoparticles prepared using 2-propanol as a grafting solvent (SPH-4P) was 303 m²/g, but the BET surface area of the aerogels prepared using toluene as a solvent was only 252 m²/g and lower (FIG. 14). Furthermore, the pore volume was larger in the former compared with that of the latter (FIGS. 15A-15B). This may be why SPH-4P could accommodate a larger amount of DEX than did SPH-4, although the rate by which the former could adsorb the DEX molecules was lower.

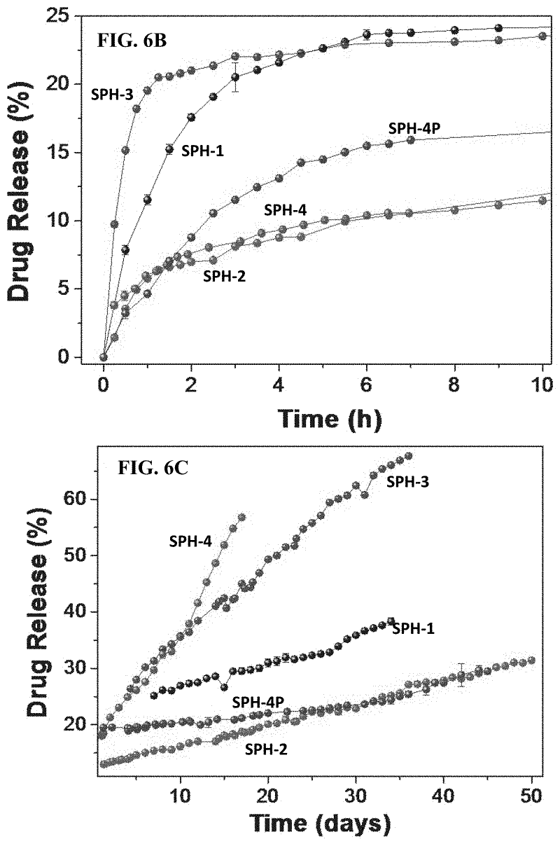

The drug release profiles of all the DEX-saturated SPH aerogels in PBS (pH 7.4) at 37° C. featured an initial burst release of DEX molecules in the first few hours and a slower release in the later hours (FIGS. 6A-6C). This burst release was probably related to the DEX molecules residing outside the pores of the mesoporous silica nanoparticles in the aerogels since the drug molecules trapped within the pores of the particles within the aerogels would likely to take longer times to make it into the solutions. A more pronounced initial release was observed for SPH-1 and SPH-3, with ca. 25% of DEX being released within 6 h, which was most likely due to their smaller proportion of polymer. In contrast, only ca. 10% of DEX was released in 6 h from SPH-2 and SPH-4, whose proportion of polymer was greater.

The release profiles of DEX from the aerogels over the course of longer time periods are shown in FIG. 6A. The percentage of DEX released by the aerogels and the drug's release kinetics were found to vary substantially from aerogel to aerogel. The release of DEX from SPH-4 was the first to reach equilibrium, unleashing a total of 60% of DEX in 15 days. Higher percentages of DEX than this amount were released by some of the other aerogels over times longer than 15 days, with the amounts slightly varying from aerogel to aerogel. For instance, a total of 70% of DEX was released by SPH-3 in 40 days and a total of 64% of DEX was released by SPH-1 in a longer time (or in 50 days). However, the release of DEX from SPH-2 and SPH-4P was smaller with an overall amount of only ca. 30% being released by them after 60 days. The ability of the aerogels to release the payloads of DEX molecules over long time was most likely due to the fact that most of the drug molecules were held up deep within the hydrophobic pores of the mesoporous silica nanoparticles embedded within the polymer. As a result, the DEX molecules could diffuse out only slowly, especially compared with those present within the polymer matrices outside of the mesoporous silica nanoparticles. Besides the pores of the mesoporous SBA-15 nanoparticles within the aerogels, the PVA-to-PAA ratio used to make the materials also hugely governed the release profiles of the drug (or how long it would take for the DEX molecules to release). In the time range of about 5 to 40 days, the slowest release of DEX was exhibited by SPH-1 and SPH-2, which were made from a PVA-to-PAA ratio of 1:1, whereas the fastest release was exhibited by SPH-3 and SPH-4, which had a PVA-to-PAA ratio of 3:1. These differences should be the result of the types of porous structures the materials possessed, as seen in the SEM images in FIGS. 3 and 12, and the structural differences were, in turn, due to the different PVA-to-PAA ratios in their precursors. The well-aligned channel-like pores present in SPH-3 and SPH-4 could be expected to facilitate the release of DEX, whereas the randomly distributed and more compacted pore structures of SPH-1 and SPH-2 could hinder the release of DEX.

These release patterns did not correlate with the extent of water uptake by the materials though, which were higher for the aerogels possessing lower density, i.e., SPH-1 and SPH-3. These two materials displayed a pronounced initial burst release of DEX molecules for about 2 days, which were most likely released from the polymer phase or outside of the pores of the mesoporous silica nanoparticles. This was supported by the dependence of the burst release rate on the bulk density of the aerogels, where a more pronounced burst release was seen from the aerogels possessing lower bulk densities. This can be rationalized based on the fact that the materials with low bulk density would have larger exposed area for the drug molecules to be quickly released from or through. Additionally, the chemical environment of the pores of the SBA-15 mesoporous silica nanoparticles was found to affect the release patterns of DEX from the aerogels, as can be seen when comparing the DEX release profile from SPH-4 versus that from SPH-4P. The fraction of DEX released by SPH-4P (i.e., ˜30% in 40 days) was half as much as the one released by SPH-4 (i.e., ˜60% in 40 days). SPH-4P material, whose mesoporous silica nanoparticles' pores were modified with methyl groups using 2-propanol as a solvent, had higher pore volume and surface area than SPH-4 (FIGS. 14 and 15A-15B). This is most likely why DEX-loaded SPH-4P showed a faster initial release of DEX in the first few days than DEX-loaded SPH-4 did. Conversely, the strong interactions between the DEX molecules and the denser hydrophobic surfaces of the mesoporous silica nanoparticles in SPH-4, or the stronger adsorption of these hydrophobic molecules in this material, could make the DEX molecules to remain trapped within the pores of the mesoporous silica nanoparticles and unable to come off quickly. The considerable faster rate of release of DEX by SPH-1 in times of 35 to 50 days was also noteworthy; this might be due to the combination of the two factors governing the release of DEX: the PVA-to-PAA ratio used to make the material and the density of the material. SPH-1 was obtained from a precursor with lower PVA-to-PAA ratio and had lower bulk density compared with the other materials studied here.

Drug release profiles by different materials can be analyzed by various models and methods. The complex drug release profiles by the hybrid materials in the present case were fitted well using the Korsmeyer-Peppas and zero-order model (Korsmeyer, et al., 1983, Int'l J. Pharmaceutics 15:25-35; Yu, et al., 2013, Cellulose 20:379-389). First, the release profiles of the drugs were divided into two stages: an initial, fast release stage and then a retarded release stage. The data points and release profile for the initial release stage are shown in the FIG. 6B and the points considered for the slow release stage is presented in the FIG. 6C. The initial release stage was modeled based on Korsmeyer-Peppas model. The retarded release stage was modeled with a zero-order release kinetic equation. The obtained parameters are presented in Table 4.

The diffusion exponents for the initial release stage for materials SPH-1 and SPH-3 show an anomalous transport process, indicating that the release process is partially influenced by the relaxation of the polymer chains in these two materials. It is worth noting here again that SPH-1 and SPH-3, which were prepared from precursors with relatively low S/L ratios, have relatively low density and more branched polymer chains, which means that the polymers have higher degrees of freedom to undergo conformational changes during the initial drug release processes. This result is also in agreement with the water uptake capacity of the materials, which was higher for both SPH-1 and SPH-3. On the other hand, the hybrid materials prepared from precursors with higher S/L ratio, SPH-2 and SPH-4, are denser and have more rigid matrices; consequently, the initial diffusion process in these materials is not affected by the possible relaxation of their polymer matrices as much.

TABLE 4
Diffusion Exponents (n) and diffusion mechanisms determined
Korsmeyer-Peppas and zero-order model for the slow drug.

| | | | | Retarded | |
|---|---|---|---|---|---|
| | Initial Release Stage ^{a} | | | Release Stage ^{b} | |
| Sample | n | R² | Diffusion Mechanism | K (μg · h⁻¹) | R² |
| SPH-1 | 0.55 | 0.99 | Anomalous Transport | 0.76 | 0.96 |
| SPH-2 | 0.30 | 0.98 | Quasi-Fickian | 0.64 | 0.99 |
| | | | Diffusion | | |
| SPH-3 | 0.57 | 0.99 | Anomalous Transport | 2.18 | 0.99 |
| SPH-4 | 0.32 | 0.99 | Quasi-Fickian | 3.88 | 0.98 |
| | | | Diffusion | | |
| SPH-4P | 0.70 | 0.98 | Anomalous Transport | 0.35 | 0.92 |

^{a} Analyzed using the Korsmeyer-Peppas model and the data points shown in FIG. 6B.
^{b} Analyzed using the zero-order model and the data points shown in FIG. 6C.

TABLE 5
Surface area and pore volume of the SPH aerogels
synthesized and studied.

| | | Pore Volume |
|---|---|---|
| Conditions | Surface Area (m²/g) | BJH Desorption (cm³/g) |
| SPH-1^{a)} | 0.04 | — |
| SPH-2^{a)} | N/A | — |
| SPH-3^{a)} | 0.17 | — |
| SPH-4^{a)} | 0.20 | — |
| SPH-5^{a)} | 0.02 | — |
| SPH-6^{a)} | 0.02 | — |
| SPH-4P^{b)} | 0.03 | — |
| SBA-15^{c)} | 252.00 | 0.42 |
| SBA-15^{d)} | 303.00 | 0.59 |

^{a)}These aerogels (SPH-1 to SPH-6) contain mesoporous silica nanoparticles whose internal pores are functionalized with HMDS using toluene as a solvent.
^{b)}This aerogel contains mesoporous silica nanoparticles whose internal pores are modified with HMDS using 2-propanol as a solvent.
^{c)}SBA-15 mesoporous silica nanoparticles whose internal pores were modified with HMDS using toluene as a solvent.
^{d)}SBA-15 mesoporous silica nanoparticles whose internal pores were modified with HMDS using 2-propanol as a solvent.

For the retarded release stage, the drug release process from all materials can be fit with a zero-order release model with excellent R² values. In a zero-order release, the drug is released at a constant rate represented by the release kinetic constant (K). Drug delivery systems with zero-order kinetics are often sought for controlled release of many types of drugs, including DEX. It is observed that the release kinetic constant varies from 0.352 to 3.88 μg.h⁻¹ depending on the composition and structural makeup of the hybrid materials reported. It is worth noting that the values of release kinetic constant of SPH-1 and SPH-2 are lower than those of SPH-3 and SPH-4; this suggests that the release kinetic constant of the drug in these hybrid materials varies depending on the PVA/PAA ratio used to make the materials. In other words, the release kinetic constant is low for the materials prepared from 1:1 ratio of PVA/PAA; conversely, the value is high for those prepared from 3:1 ratio of PVA/PAA. Thus, a higher release kinetic constant of the drug can be correlated to a more hydrophilic feature of the polymeric matrices, which is observed for the materials made from 3:1 ratio of PVA:PAA (i.e., SPH-3, and SPH-4).

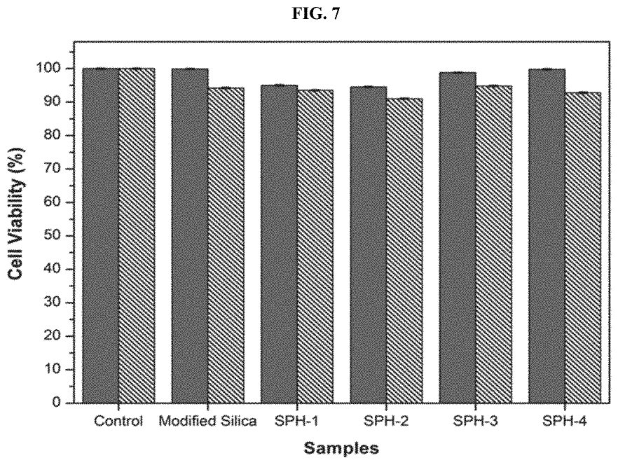

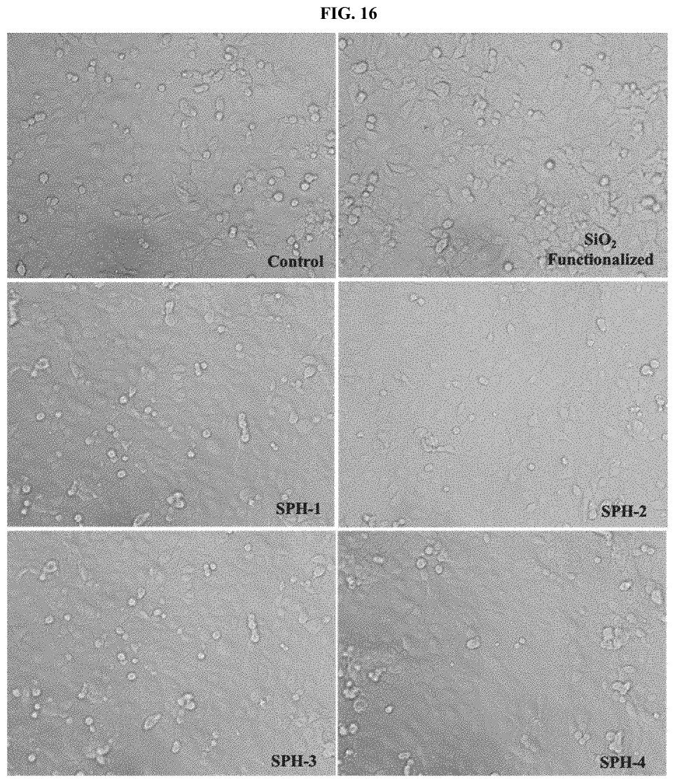

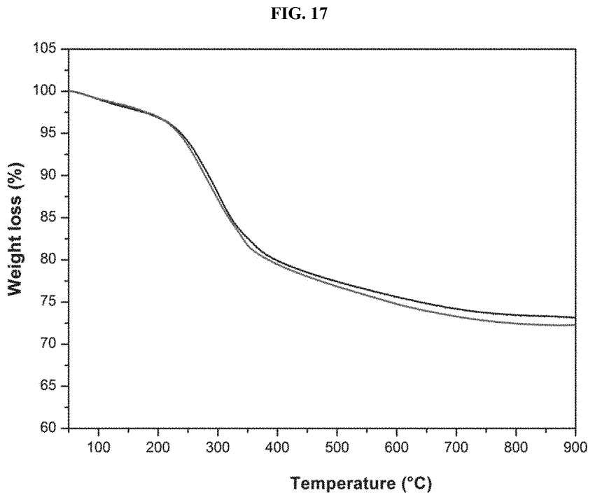

To gain valuable insights about the potential of the new aerogels for medical applications, their biocompatibility/cytotoxicity and ability to promote cell growth assays were evaluated (FIG. 7). Low/no cytotoxicity and high proliferation are two important criteria for a material to be potentially suitable for medical applications. The biocompatibility/cytotoxicity and cell proliferation of SPH-1, SPH-2, SPH-3, SPH-4, and organic-modified silica materials were studied by using Vero cells and L929 fibroblasts, respectively. Cell growth and cell viability of all the materials was analyzed using the MTT assay after keeping the samples in contact with cell culture media for 72 h. The control experiment for cell growth was carried out in the absence of samples (on polystyrene well-plates). The cell viability results for the aerogel materials versus a control with Vero cells (red bar) and L929 cells (line bar) after incubation for 72 h (FIG. 7) showed no substantial differences. Based on the data (the red bar in FIG. 7), the aerogels barely caused changes in cell viability, as the cell viability was ca. 100% in case of SPH-3 and SPH-4 and it decreased by only ca. 5% (with respect to that of the control) in the case of SPH-1 and SPH-2. Images obtained by optical microscopy for a typical population of Vero cells grown on the surface of 96-well plates after a period of 72 h in the presence of the samples (FIG. 16) corroborated the cell viability results obtained by the MTT assay above. The images further showed that the cells maintained their morphologies when compared with those of the untreated cells (the negative control samples). These results indicated that the aerogel materials reported in this work did not shown cytotoxicity directly or indirectly toward Vero cells. In the case of the organic-functionalized silica nanoparticles, where three different concentrations (50, 100 and 200 μg/mL) were incubated with the cells for 72 h, all the concentrations, including 200 μg/mL, showed a cell viability of ca. 100%, the same value as that of the control (FIG. 7).

The results of the cell proliferation experiments (line bars in FIG. 7) did not also show much difference with respect to the control experiment, as the cell viability in the presence of the aerogel samples did not differ significantly from that of the control. The aerogel samples all showed cell viability higher than 90% (ca. 93.5% on average). L929 fibroblasts are popular cell lines used as an in vitro model and tool in several standard assays, e.g., for testing the biocompatibility of materials. Furthermore, materials that result in similar cell proliferation properties as the control have the potential to regenerate skin. So, the results obtained on cell growth over the new 3D aerogels reported here and their biocompatibility with good cell proliferation can make them highly suitable for use as a prolonged and sustained drug delivery system in biological environments, e.g., for skin treatment. This is true especially since the materials are proven to hold the anti-inflammatory drug DEX and release the payload of the drug over long periods of time. Besides, thanks to their quaternary groups, the functionalized mesoporous silica nanoparticles in the materials showed antibacterial activity against three different bacteria, E. coli, S. aureus, B. subtilis, and P. aeruginosa (see Table 2 for details). These results suggest that the aerogels have a potential to render antibacterial activity while serving as controlled release systems for drugs from wound dressings.

As demonstrated herein, novel hybrid aerogel materials for biomedical applications have been synthesized by combining SBA-15 mesoporous silica nanoparticles with hyperbranched polymer networks comprising PAA and PVA. The synthetic method has allowed water-stable aerogels with controlled structures and well-aligned pores to form, especially in cases where relatively higher amounts of PVA were used to synthesize the materials. By changing the relative amount of the mesoporous silica nanoparticles, the PAA-to-PVA ratio, the solid-to-liquid (S/L) ratio and the density of functional groups on the internal surfaces of the mesoporous silica nanoparticles, aerogels with different bulk densities and adsorption and prolonged release properties for a hydrophobic drug have been synthesized. The resulting hybrid aerogel materials have been demonstrated to serve as ideal host materials with high adsorption capacity for a model hydrophobic drug, DEX, exhibiting prolonged, sustained release profiles for it, for as long as two months. Both components of the hybrid aerogels (i.e., the polymers and mesoporous silicas nanoparticles) have been found to be responsible for these properties: while their organic-functionalized mesoporous silica nanoparticles have allowed the aerogels to have a high loading of DEX, their polymer matrices have provided physical stability and slow and prolonged release profiles for the adsorbed DEX molecules. The incorporation of mesoporous silica nanoparticles within hyperbranched polymer aerogels has helped hydrophobic drug molecules to be hosted with chemically modified internal pores of trapped nanoparticles. This synthetic approach can be used to make other effective drug delivery systems for hydrophobic drugs that are difficult to deliver otherwise. The aerogels may potentially serve as delivery systems for hydrophilic drugs as well, since the inner walls of the silica present in these materials can be easily tailored with hydrophilic groups. The in vitro cytotoxicity and cell proliferation results showed that the aerogels had no toxicity for Vero cells and displayed a good cell proliferation for L929 cells. Furthermore, the silica nanoparticles intentionally functionalized (externally) with quaternary groups and dispersed into the aerogels showed good antibacterial activity, which is important for applications such as wound treatment. The results overall have indicated that the aerogels could be used as drug carriers in biological environments, especially as sustained delivery systems for drugs, for long periods of time for potential applications, such as wound care dressings, or among other things. Besides, additional functional groups (e.g., antibodies to target tissues) can easily be tethered onto such aerogels to further tailor their properties and extend their biological/medical applications. This also means that the materials can serve as multifunctional drug delivery systems that can meet the long list of requirements for topical applications in very special cases, such as treatment of severe skin burns and melanomas.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A composition comprising poly(vinyl alcohol) (PVA), poly(acrylic acid) (PAA), and at least one mesoporous silica nanoparticle.

2. The composition of claim 1, wherein the PVA and the PAA are at least partially polymerized.

3. The composition of claim 2, wherein the at least partially polymerized PVA and PAA form a hyperbranched polymer network.

4. The composition of claim 2, which is essentially free of solvents or liquid components.

5. The composition of claim 4, which is an aerogel.

6. The composition of claim 2, wherein the at least one mesoporous silica nanoparticle is dispersed within the polymer.

7. The composition of claim 1, wherein the at least one mesoporous silica nanoparticle comprises a SBA-15 mesoporous silica nanoparticle.

8. The composition of claim 1, wherein the at least one mesoporous silica nanoparticle is derivatized with at least one functional group.

9. The composition of claim 8, wherein the at least one functional group comprises a hydrophobic group or a quaternary ammonium group.

10. The composition of claim 8, wherein the hydrophobic group comprises a hydrocarbyl, tri(hydrocarbyl)silane, halogen, or thiohydrocarbyl group, wherein each group is optionally substituted with at least one selected from hydrocarbyl, tri(hydrocarbyl)silane, halogen, and thiohydrocarbyl.

11. The composition of claim 8, wherein the hydrophobic group is located within a pore of the nanoparticle.

12. The composition of claim 8, wherein the quaternary ammonium group is located on the outer surface of the nanoparticle.

13. The composition of claim 8, further comprising a therapeutic agent that is associated with the at least one functional group.

14. A method of treating a disease or disorder in a subject, the method comprising administering to the subject an aerogel composition comprising a co-polymer of poly(vinyl alcohol) (PVA) and poly(acrylic acid) (PAA), wherein at least one mesoporous silica nanoparticle is dispersed within the co-polymer and is derivatized with the at least one functional group.

15. The method of claim 14,
wherein the at least one functional group is a quaternary ammonium salt and is on the outer surface of the nanoparticle, and
wherein the disease or disorder comprises bacterial infection.

16. The method of claim 14,
wherein a therapeutic agent is associated with the at least one functional group, and
wherein the therapeutic agent treats the disease or disorder.

17. The method of claim 16,
wherein the at least one functional group is hydrophobic and is within a pore of the nanoparticle, and
wherein the therapeutic agent is hydrophobic.

18. The method of claim 14, wherein the aerogel composition is administered through a route selected from the group consisting of oral, parenteral, transdermal, transmucosal, intravesical, intrapulmonary, intraduodenal, intragastrical, intrathecal, subcutaneous, intramuscular, intradermal, intra-arterial, intravenous, intrabronchial, inhalation, and topical.
